SEQUENCE LISTING (37 sequences)

SEQ ID NO: 1 - DNA, 2957 bp, Physcomitrella patens
  misc_feature (432)..(432): n is a, c, g, or t
agttgtcgatttgttgtttttgatatgtaaggcggttgccttcgcgccgtgcttgattgtaattgtaattcaatctggagtgtgagatatatatatatatatatatatagcgagagggagagagaaagagagagagagggagagagaaagagagagagagggagagagagagatggcttgtgtatgagggccatgcgaggaggaggctgtgtttgttgcccgaagagatgggatggtttatgtgtagtgcaggggttggatgtgaagcacctgtttgaaggagtctgcgagagtttgaaattcggattcagagtgcggcgatcgatggtgcaacgttgttagcagtgattgttttcgccaacagaactgacatcatttggattttttttacgcgtggatgtgccctctttttaaaaaatttccgcgtggaanagagacgggggtttgtaatggaggcaggctgtggtcatcacccctagtatagcctgtcaagagagttcaaattcggtaatatgaagagggggtcgagactaccggatatggcgtgtacagggcggcaaagaaatgatcttatcctagttgcaattgtttgcttgttttttatggtgatattcatcccaccatatctccaaatgaactcacttccggacattgattctcctgattcggacaagaaatcatcaagctactcgaaaaaaaccactctagaagccaatagtaaggaggaacgccgtagtccggggaataccacaggcgacattgtttctctggatgatgtgatagatcgtgcctggtctgctggtgccaaagcgtgggaagaactggaaactgcgttaagaaatggagaaggtgtctcaaagaatgtcagtaatgccactgcaaatgctgatccgtgtccagcatcactctctgcagcagggaaaaagttagacgaattgggtaaagtcttccccttgccctgtggtctaatgtttgggtcagccattactctgattggaaagcctcgagaggctcacatggagtacaaaccgccaatcgccagagttggggaaggcgtctctccatatgtcatggtttcccagttcttagtagagttacaaggcttaaaggtggtgaaaggtgaagatcctcctcgaattctacacttgaatcctcgacttcgtggtgattggagctggaaacccatcattgagcacaacacttgttatcggaaccagtggggtcctgcccaccgatgcgagggttggcaagtgcctgaatacgaagaaactgttgacggtcttcccaagtgcgagaagtggcttcgagatgatggcaagaaacctgcttcaacgcaaaaatcttggtggcttggaagattagttggtcgttctgacaaggagacgcttgaatgggagtacccattatctgagggtcgggagttcgttctcaccattcgagcaggtgttgaagggtttcatgtgactatcgatggtcgtcacatcagctcgtttccttatcgtgtgggttacgctgtggaagaaacaacggggatattagtagcaggagacgttgatgtgatgtctatcacagtgacatccctacccttaacacatcctagctactaccctgagttagttttggaatcgggggacatttggaaggcaccacctgtcccagctaccaagatagatttatttattgggatcatgtccagcagtaaccattttgcagaacggatggcagtaaggaagacgtggtttcaatctaaagctattcaatcttcgcaggccgtggctcgcttctttgtagctctgcatgcaaacaaggatatcaatatgcagttgaagaaggaggcagactattatggcgatattataatcctgcctttcatcgacagatatgatatagtggttctcaagaccgttgaaatttgcaagtttggggtccagaatgtcacagctaagtatattatgaagtgtgacgatgacacttttgtgaggattgatagcgttctcgaagagattcgaactacttcaatatcacaaggcctttacatgggtagcatgaatgagtttcacaggcctcttcgttctggaaagtgggccgtgactgccgaggaatggcctgagcgaatttacccaatatatgctaatggaccaggatatatcctgtcagaggatattgtgcatttcattgtggagatgaatgagagaggcagtttgcagttatttaagatggaggacgtcagtgttggaatatgggtacgcgaatatgcgaagcaagtgaagcacgttcaatacgaacatagcatacggtttgctcaagccggttgtataccgaaatacttgacagctcattaccaatcgccgcgtcaaatgctgtgtctgtgggacaaggtacttgctcatgacgatgggaaatgctgcaacttgtgaggaaaatacatacaatgaatgtgttcaacggtctttaccagacagaattactttgggtcgggaaccagatatagcagacagctcacattcaattcagccgtgttgatccagaggggtaattgatagtttccttgtcccctaccctctctagaggtggagatcttacaacttaatcaaatgatcctctgcaatgtcacttgtcacaatacttagtatagctcaaaattggccacggatattcaggaatgttcatcttgtaaggtcgcagcttgtgagtaaatggttgggtggtgtcgatggcatggttgcttatcaatccctcttagcatcagtgatcgtcagaatcagtgttttcgacactccccggtggagtattttttcgattctcttgattccactcaagtggtactagcttatatttagtgaggcctggaacccaagtagttagttcagtacgtctgccttttgccgaaatgagtagagtaatttgtggcagtagttggtgaagagacatggttaggatttagtgttcaaaatctg

SEQ ID NO: 2 - DNA, 1902 bp, Physcomitrella patens
atgaagaggggtgtgagaccaccgggtgtgggatgtacagggcggcaaagaaacaatctaatcatagtggcaatcatatgtttggtttttatagcgatattcatcccaccgtttcttgaaatgaattcacttcccgatattgattcccctgttttggagaagaaagtatcaagctatttgaaaaaagtcactctggaaacttacagtaaagaggaacgccgtagtccagggaacacaacaggtgacattgtttcgctggaagatgtgatagatcgcgcctggtctgccggcgccaaagcttgggaagagctggaaattgcattcagacagggagaacatttttcgaagaaggacaataatgccaatgcaactgcagatccatgcccagcatcactctttacaacaggaaaggaattggacaatttaggaagggtcttcccactgccttgtggtctaatgtttggatcagccataactctcattggaaagccacgggaagctcacatggagtacaaaccgccaatcgccagagttggggaaggtgtctctccatacgtcatggtgtcccagttcataatggagttacagggcttgaaggtggtaaaaggtgaagatcctcctagaatcctccacataaaccctcgactccgtggtgactggagctggaaacccatcattgagcataatacatgctatcgaaaccagtggggcccagctcatcggtgtgaaggttggcaagtacctgaatacgaagaaaccgtggacggtcttcccaagtgcgagaagtggcttcgaggcgatgacaaaaaacctgcttcgacccaaaaatcctggtggcttgggcgattagttggtcattccgacaaggagacgcttgaatgggagtatccattgtccgaaggtcgggagtttgttctcaccattcgagcaggtgtagaaggatttcacttaactattgatggtcggcacatcagttcgttcccttatcgtgcgggttatgctatggaagaagcaacaggaatatcagtggcaggagacgtcgatgttctttcgatgacagtaacatcattacctttaacacatcccagctactaccctgagttggttttggattcgggtgatatctggaaggcaccacctttaccaacaggcaagatagagttatttgttggaatcatgtcaagcagcaatcactttgcagaacgtatggcagtaagaaagacgtggtttcagtctctggttatccaatcctcccaagcggtggctcgcttctttgtagctctgcatgcaaacaaggatatcaatctgcagctgaagaaagaggctgactattacggcgatatgataattttacctttcatcgacagatatgatatagtggttcttaagaccgttgaaattttcaagtttggggtccagaatgttacagttagccacgtcatgaaatgtgacgatgacacatttgtaaggattgacagcgttcttgaagagattcgaacgacgtcagtaggacagggcctttacatgggcagcatgaatgagtttcatagaccccttcgttctgggaagtgggccgtgacagttgaggagtggcctgagcgcatttacccaacatacgcaaatggtccaggatacatcctttcggaagatattgtgcattttatagtggaggagagcaaaagaaataatttgaggttatttaagatggaggacgtcagcgtaggtatatgggtacgcgagtatgcaaagatgaagtacgtgcaatacgagcatagcgtacggtttgctcaagccggttgtatacctaactacctgacagcgcactatcaatcgccgcgtcaaatgctgtgtctgtgggacaaggtgcttgctaccaatgacggcaagtgctgcaccttgtga

SEQ ID NO: 3 - DNA, 6187 bp, Physcomitrella patens
agttgtcgatttgttgtttttgatatgtaaggcggttgccttcgcgccgtgcttgattgtaattgtaattcaatctggagtgtgagatatatatatatatatatatatagcgagagggagagagaaagagagagagagggagagagaaagagagagagagggagagagagagatggcttgtgtatgagggccatgcgaggaggaggctgtgtttgttgcccgaagagatgggatggtttatgtgtagtgcaggggttggatgtgaagcacctgtttgaaggagtctgcgagagtttgaaattcggattcagagtgcggcgatcgatggtgcaacgttgttagcagtgattgttttcgccaacagaactgacatgtaatgaatagtttcgaggcatgatcgcggtttttctcaatttgaaggggttgtttgtgggtgatctatgtgcagaagtgtcactgatggtcagattcgatgcttgacaatttgatcctttgtgagtgtgcagcatttggattttttttacgcgtggatgtgccctctttttaaaaaatttccgcgtggaaaagagacgggggtttgtaatggaggcaggctgtggtcatcacccctagtatagcctgtcaagaggtgagattgacaccctctttgctcaattgtagatttttttccttctcagggctgaatcccagtttttttttttttttttttttttttttccttcttcttcaacttcgagttcgtgtctgtatgaagaagtccacgggttcaatgtgttaagacttaggcatttccttcagctttgcctagtggagatatgcgtattttttgattgtgaggattccggttcttagaccatgattggtttattacagtggtcattcaaatcctatttgatttgagaatgtatttacttcgttgtgttgggagatgattgttccctcgaattctatgcggtagctaccgcttctttcgtaatgaagacctttgaagttcacatagacttcaagaagaatgctatttgtgtttttgtgattgtgtgttcaagtttggtgcagtattgttaaaatttgggtgatgactaagtacactttatgcggcccaagtagtcaagttgagcatttgtaaatgctgaaatgagttaggctgacggtaaatgtctgtggatgtagcctagtgatgtatttgatctcggcataatcttcagtgatcaatacaaataattcaagaaagaggggtcaatgtgttcctgcgagtaccttcgcatgttcaacgtgaactgaattatgttaattaagctgagcaacatagaccttcttgctgttgacagagttcaaattcggtaatatgaagagggggtcgagactaccggatatggcgtgtacagggcggcaaagaaatgatcttatcctagttgcaattgtttgcttgttttttatggtgatattcatcccaccatatctccaaatgaactcacttccggacattgattctcctgtcgagaagctagaagatgatgatgatgctgtcttcacttctcatagacgtcgtaaccaagagcagatttcagttgtcactgacagtggtcagagacggacagttatgccatcttcgactggtgcggaggacgtaacgaatgcaccgtctaaagattcacaggttagaccaaaagtagttgacctgaaatgcatgtggtaatcaagcactcttgtccttattcgagcttttatttcttgccatcaggtatttttaatacttccctagtgtacgctgagtgtctacattgtgtattgaatgttccttagaattgtttgtttgtttatgtttttatttttatatttctgccggctattgaggaagaatacattcaaattgttcaggattcggacaagaaatcatcaagctactcgaaaaaaaccactctagaagccaatagtaaggaggaacgccgtagtccggggaataccacaggcgacattgtttctctggatgatgtgatagatcgtgcctggtctgctggtgccaaagcgtgggaagaactggaaactgcgttaagaaatggagaaggtgtctcaaagaatgtcagtaatgccactgcaaatgctgatccgtgtccagcatcactctctgcagcagggaaaaagttagacgaattgggtaaagtcttccccttgccctgtggtctaatgtttgggtcagccattactctgattggaaagcctcgagaggctcacatggagtacaaaccgccaatcgccagagttggggaaggcgtctctccatatgtcatggtttcccagttcttagtagagttacaaggcttaaaggtggtgaaaggtgaagatcctcctcgaattctacacttgaatcctcgacttcgtggtgattggagctggaaacccatcattgagcacaacacttgttatcggaaccagtggggtcctgcccaccgatgcgagggttggcaagtgcctgaatacgaagaaactggtgagtgctgattccaccgcaccagtttgtgttttttatgctgacactatgcttctcaggtttgtagacgttaagagctgtgtaggttccgtggtacttcgaattggcacttgccacttctctcattgtaagttggtaaatgtctgcatgagcaataaattccaacactggatgtgtattttctgaaatgattcgttttcttgtagttgacggtcttcccaagtgcgagaagtggcttcgagatgatggcaagaaacctgcttcaacgcaaaaatcttggtggcttggaagattagttggtcgttctgacaaggagacgcttgaatgggagtacccattatctgagggtcgggagttcgttctcaccattcgagcaggtgttgaagggtttcatgtgactatcgatggtcgtcacatcagctcgtttccttatcgtgtggtaagttgaaaatgctatgttaacatataatgctaaagttgacctcatgtctttcttttttctttttttcttttttattttctggagggggggggggtaatgcaaatcaactctaaaattttagtataccagttaaattattcatttcaaatataacaatacaaatacacatctttttaatttgtattttttgatccctctcctcctctactaaaattaataatatagcaacattttggtactacgaaagttcatttgtattgcttcatgtcgaagatttattcaaaatttctatccctcgtgtttctgaattacattatcaacaatggaataacaataatgacggccccatccttcagacaccaggaacattacctataccagactacgtctgggtaagtctgaagaattaattataaccaagaaactagttgtattcactgtttttctttttacgcccatgcgatttatcgaagtcttcttcaatttcttattattcttctttattattttaagtttttaattatttttaaagcaacgaattgataaataaataacatattaatgtttttaactttaaagtttttttcccgtatttagtataagatttcgtcaaaacgattaggtgattagatcgaacattatctaattgcactctacttatatgatatgaagagtaatttctcttagcagaagctacatcctgctatttccttgggaaacccgattaggtctttcaaatcacccctgcttcctctataagtgtaccatgattgaggttcgttagggcattagtttaagggtatcgttgtgatgtgtgtctagttagtcttaaaatctgtgcaaatcgattcattaacaactcttttctgtagtgttttgttttgagaactgctatttatcttccattgtgcagggttacgctgtggaagaaacaacggggatattagtagcaggagacgttgatgtgatgtctatcacagtgacatccctacccttaacacatcctagctactaccctgagttagttttggaatcgggggacatttggaaggcaccacctgtcccagctaccaagatagatttatttattgggatcatgtccagcagtaaccattttgcagaacggatggcagtaaggaagacgtggtttcaatctaaagctattcaatcttcgcaggccgtggctcgcttctttgtagctctggtacttcctcctatcaaatctcattaactttcgaattattagtgatcatctacataagtggtctgttgattgctgaaaggtggctgttgcgtgcctttgcgtaatgactttccaaattcatttagaacagtggaaacataatttgtgtgttgcgttgcgtatttaactttttcggtgaatgtcttattgaattgtgatgtagcatgcaaacaaggatatcaatatgcagttgaagaaggaggcagactattatggcgatattataatcctgcctttcatcgacagatatgatatagtggttctcaagaccgttgaaatttgcaagtttggggtacgtgtgtcgaataatggcttcaaagctttgtgacggtgtctgcaatttggggatggtgataatgaggcttgataccaactgaaggttaggtgacttttaacactaggttctgcttactgtgcaggtccagaatgtcacagctaagtatattatgaagtgtgacgatgacacttttgtgaggattgatagcgttctcgaagagattcgaactacttcaatatcacaaggcctttacatgggtagcatgaatgagtttcacaggcctcttcgttctggaaagtgggccgtgactgccgaggtatttttatttttatttttggcttttgtcgggaacgtgagagaaaccaagatgaatataatcacgatgttgttttttattgcaaggatttatttgatgctcttgagaaatctgtggtagccataccactcaatttggatactagatgtgttcgtccttatgtataaaaatgaaacatgtgcttttcaggaagattaattcagtttgacttgtacgtctagttagattgatggtgatgaaacaagaggattatctcgcgaattgacaagtgggttgcttggacaggaatggcctgagcgaatttacccaatatatgctaatggaccaggatatatcctgtcagaggatattgtgcatttcattgtggagatgaatgagagaggcagtttgcaggtaggttcttttagaactgtgtcgtcgctattacacgtctacaagttttaaaaattagaaactttcttgttggcaaatttccatccaggaatctttttgcaccgcaagttcgtaataggagtcggtacattctgtgtgtgtgcatcgtttgttaaatgcatttttcaattttcttttgcttaaaatatctctgttgtcgatatctcctcatgatcttgcattgtgaacatgagaagatatgaaatgtgaactcaatattcttctatgatcatgtgcagttatttaagatggaggacgtcagtgttggaatatgggtacgcgaatatgcgaagcaagtgaagcacgttcaatacgaacatagcatacggtttgctcaagccggttgtataccgaaatacttgacagctcattaccaatcgccgcgtcaaatgctgtgtctgtgggacaaggtacttgctcatgacgatgggaaatgctgcaacttgtgaggaaaatacatacaatgaatgtgttcaacggtctttaccagacagaattactttgggtcgggaaccagatatagcagacagctcacattcaattcagccgtgttgatccagaggggtaattgatagtttccttgtcccctaccctctctagaggtggagatcttacaacttaatcaaatgatcctctgcaatgtcacttgtcacaatacttagtatagctcaaaattggccacggatattcaggaatgttcatcttgtaaggtcgcagcttgtgagtaaatggttgggtggtgtcgatggcatggttgcttatcaatccctcttagcatcagtgatcgtcagaatcagtgttttcgacactccccggtggagtattttttcgattctcttgattccactcaagtggtactagcttatatttagtgaggcctggaacccaagtagttagttcagtacgtctgccttttgccgaaatgagtagagtaatttgtggcagtagttggtgaagagacatggttaggatttagtgttcaaaatctg

SEQ ID NO: 4 - DNA, 4087 bp, Physcomitrella patens
atgaagaggggtgtgagaccaccgggtgtgggatgtacagggcggcaaagaaacaatctaatcatagtggcaatcatatgtttggtttttatagcgatattcatcccaccgtttcttgaaatgaattcacttcccgatattgattcccctgtgtataggttagaaggtattaacttcgcttcacatagacgtcgctatcaagaacaggattcacgtgtcagttacagtggctatggacagccagatatgccatcaactggtgatgaagacataacgaagacaccgtctaaagcttcacaggttagtgcagaaatgattggttcgccctcgctatgccagtcaggcttactgagttctacttggatcgttctacttggatcttttatggcttcctagcagtcggaggtttctttctggtttgaagaaagccatgtatggaacgtttacaggttttggagaagaaagtatcaagctatttgaaaaaagtcactctggaaacttacagtaaagaggaacgccgtagtccagggaacacaacaggtgacattgtttcgctggaagatgtgatagatcgcgcctggtctgccggcgccaaagcttgggaagagctggaaattgcattcagacagggagaacatttttcgaagaaggacaataatgccaatgcaactgcagatccatgcccagcatcactctttacaacaggaaaggaattggacaatttaggaagggtcttcccactgccttgtggtctaatgtttggatcagccataactctcattggaaagccacgggaagctcacatggagtacaaaccgccaatcgccagagttggggaaggtgtctctccatacgtcatggtgtcccagttcataatggagttacagggcttgaaggtggtaaaaggtgaagatcctcctagaatcctccacataaaccctcgactccgtggtgactggagctggaaacccatcattgagcataatacatgctatcgaaaccagtggggcccagctcatcggtgtgaaggttggcaagtacctgaatacgaagaaaccggtgagtgctggttccatcacactttatcttttcatagtgacacggttctttttaggtgtactagtgttgaaagctgtgcatgttaaatggtaaccctaatcaatcttctcgctaattttcgcattgcaaggtctccgctgcttggacaatcagcactctaacattggctgtatttactgaaatgattctttactttgtagtggacggtcttcccaagtgcgagaagtggcttcgaggcgatgacaaaaaacctgcttcgacccaaaaatcctggtggcttgggcgattagttggtcattccgacaaggagacgcttgaatgggagtatccattgtccgaaggtcgggagtttgttctcaccattcgagcaggtgtagaaggatttcacttaactattgatggtcggcacatcagttcgttcccttatcgtgcggtgagttgaaaatactagtttgatatctaatgatgaggtttaccgcaggtatatttggtctcattgtcaagtgtgtgtgtgtgtgttgtttttcttttttccttttcattttctgaatcataatgataagaaatcaattctatgaaacttagcgtcaatattttaaagttttattgtttttgtttgtttttatttttttgtgttttgtgttttgtgtttatttcacaatacaatgttaacaatggaatagaaacaatgatggtcccacctcacagacaccaggtacactacctacaccagactgcgtctgagtaagtttaagaaacagcaaccaccaacaatctgattgtaaattctaaattccttctccaccagaaaaccatgtgatccgtcttgcagttctgcttgcactctacctatatgatccaaagagtaattcctcttaacaggagttataacctgctggggttttgaaaataccgatgagttcaaattgtaaacaaaccccggatctatttcaagggtatgaagggcttagctttgtttaagaataaggtcaagagtatctgtgtggtgagcatcccaaaatggatgcaaatttgttaattggcaactgttttctgtggtatgttttgtgacgcactatttattgtgtattgtgcagggttatgctatggaagaagcaacaggaatatcagtggcaggagacgtcgatgttctttcgatgacagtaacatcattacctttaacacatcccagctactaccctgagttggttttggattcgggtgatatctggaaggcaccacctttaccaacaggcaagatagagttatttgttggaatcatgtcaagcagcaatcactttgcagaacgtatggcagtaagaaagacgtggtttcagtctctggttatccaatcctcccaagcggtggctcgcttctttgtagctctggtacttgtcattatactcttttttcgtgccaagtatcgtgaactcgggaatatttaaaaagtgcaaacaacaagtgagctgttaattgctgaaaattggtgttataagtcttgatgcagtgaccttccagattgaccaagtatatcagaccttagaatttgaacagcactacttacttaccatttttaatgaatcccttgttgggttgtgatgcagcatgcaaacaaggatatcaatctgcagctgaagaaagaggctgactattacggcgatatgataattttacctttcatcgacagatatgatatagtggttcttaagaccgttgaaattttcaagtttggggtaagcgaattaaaatttgtagtatttacaaagtaatatttttaaacgttgtgaggacatctgcaacttgatatatttctttcgtgaggttcgatgctgattaaagcttaggtgatttaaaagcacggtgttgcttgctatgcaggtccagaatgttacagttagccacgtcatgaaatgtgacgatgacacatttgtaaggattgacagcgttcttgaagagattcgaacgacgtcagtaggacagggcctttacatgggcagcatgaatgagtttcatagaccccttcgttctgggaagtgggccgtgacagttgaggtaattttccctgtaccaaattatccaagattttcgtaaccattgtgtgccttattcatttcttctgaaatctcaagaaaaatgaaaaatgcttgagaaacgctcgtagccgtatcacattatgcgaattccaaaaaagaatgtggaacaaaagttcttgtgaaaataattgatatgttcaaattgtacacatttatgcactaagataagatatgtgcaaatagtgccttccagtggtctagaaaatgcttgtttttttttggaagctttaactttatttagcttgaacatcttgtttgagggttggtgaccaagtaagaaggtccatacaagacaataaatggattggttcgtgcatgtacaggagtggcctgagcgcatttacccaacatacgcaaatggtccaggatacatcctttcggaagatattgtgcattttatagtggaggagagcaaaagaaataatttgagggtgcgtttttcatagctgtgtcctggtgattaaatgccccatgttcaacattgaaaccttcatcttggacagttttccatccatgtatctcctgtcattataattgcattatagaactgttcgcgtgtacatttctttcctgttcctctttttcattttctttttctcttcttttcttcatttacttctcctcttgtcgatgctttctgttgaccttatattgtggatatgtatctcttcagtactacggagacgatatgaaacataagtttgatattcttctgtgataaagcgcagttatttaagatggaggacgtcagcgtaggtatatgggtacgcgagtatgcaaagatgaagtacgtgcaatacgagcatagcgtacggtttgctcaagccggttgtatacctaactacctgacagcgcactatcaatcgccgcgtcaaatgctgtgtctgtgggacaaggtgcttgctaccaatgacggcaagtgctgcaccttgtga

SEQ ID NO: 5 - DNA, 18 bp, Artificial
  Synthetic primer
ctgaatatccgtggccaa

SEQ ID NO: 6 - DNA, 29 bp, Artificial
  Synthetic primer
ttcgagctcatgaagagggggtcgagact

SEQ ID NO: 7 - DNA, 29 bp, Artificial
  Synthetic primer
tacgagctcatgaagaggggtgtgagacc

SEQ ID NO: 8 - DNA, 28 bp, Artificial
  Synthetic primer
gtagagctctcacaaggtgcagcacttg

SEQ ID NO: 9 - DNA, 30 bp, Artificial
  Synthetic primer
tacggatccaacttcgagttcgtgtctgta

SEQ ID NO: 10 - DNA, 33 bp, Artificial
  Synthetic primer
acactaagcttctaatcaatgtccggaagtgag

SEQ ID NO: 11 - DNA, 34 bp, Artificial
  Synthetic primer
ttagaagcttagtgtacgctgagtgtctacattg

SEQ ID NO: 12 - DNA, 33 bp, Artificial
  Synthetic primer
cattgtcgaccctacacagctcttaacgtctac

SEQ ID NO: 13 - DNA, 1585 bp, Artificial
  GalT knock-out
caacttcgagttcgtgtctgtatgaagaagtccacgggttcaatgtgttaagacttaggcatttccttcagctttgcctagtggagatatgcgtattttttgattgtgaggattccggttcttagaccatgattggtttattacagtggtcattcaaatcctatttgatttgagaatgtatttacttcgttgtgttgggagatgattgttccctcgaattctatgcggtagctaccgcttctttcgtaatgaagacctttgaagttcacatagacttcaagaagaatgctatttgtgtttttgtgattgtgtgttcaagtttggtgcagtattgttaaaatttgggtgatgactaagtacactttatgcggcccaagtagtcaagttgagcatttgtaaatgctgaaatgagttaggctgacggtaaatgtctgtggatgtagcctagtgatgtatttgatctcggcataatcttcagtgatcaatacaaataattcaagaaagaggggtcaatgtgttcctgcgagtaccttcgcatgttcaacgtgaactgaattatgttaattaagctgagcaacatagaccttcttgctgttgacagagttcaaattcggtaatatgaagagggggtcgagactaccggatatggcgtgtacagggcggcaaagaaatgatcttatcctagttgcaattgtttgcttgttttttatggtgatattcatcccaccatatctccaaatgaactcacttccggacattgattagaagcttagtgtacgctgagtgtctacattgtgtattgaatgttccttagaattgtttgtttgtttatgtttttatttttatatttctgccggctattgaggaagaatacattcaaattgttcaggattcggacaagaaatcatcaagctactcgaaaaaaaccactctagaagccaatagtaaggaggaacgccgtagtccggggaataccacaggcgacattgtttctctggatgatgtgatagatcgtgcctggtctgctggtgccaaagcgtgggaagaactggaaactgcgttaagaaatggagaaggtgtctcaaagaatgtcagtaatgccactgcaaatgctgatccgtgtccagcatcactctctgcagcagggaaaaagttagacgaattgggtaaagtcttccccttgccctgtggtctaatgtttgggtcagccattactctgattggaaagcctcgagaggctcacatggagtacaaaccgccaatcgccagagttggggaaggcgtctctccatatgtcatggtttcccagttcttagtagagttacaaggcttaaaggtggtgaaaggtgaagatcctcctcgaattctacacttgaatcctcgacttcgtggtgattggagctggaaacccatcattgagcacaacacttgttatcggaaccagtggggtcctgcccaccgatgcgagggttggcaagtgcctgaatacgaagaaactggtgagtgctgattccaccgcaccagtttgtgttttttatgctgacactatgcttctcaggtttgtagacgttaagagctgtgtagg

SEQ ID NO: 14 - DNA, 21 bp, Artificial
  Synthetic primer
tggcacgatacagtggcatga

SEQ ID NO: 15 - DNA, 29 bp, Artificial
  Synthetic primer
tggaattcattcaagaaacggtgggatga

SEQ ID NO: 16 - DNA, 27 bp, Artificial
  Synthetic primer
tgaattccataacgaagacaccgtcta

SEQ ID NO: 17 - DNA, 24 bp, Artificial
  Synthetic primer
caagcagcggagaccttgcaatgc

SEQ ID NO: 18 - DNA, 1656 bp, Artificial
  GalT knock-out
tggcacgatacagtggcatgagatttatcgctgccaaactgtggacaatgatgtttgaaacagtctattcatcactggttggcaaattctatgtacagggctaaaagggccaaactaggcttaacagcagtgatcgaggttcttgagcaggatcagcgcaagggtaaggttgcttaggaccgcttcaacctggtgagttagacactcaaaataattacgaaacagtgacatttataagctttgtgtcgtcactactttgagccttcagagtacatttataggtggtgacttcgttaatgatgttaaaaatatgaggtgaggacatgtcttcttgtgattagagtgatcactttgatccttttgcaaacgctgaaaggagtaagtctgattgtcaacagaaatgtttttggttgcagcctggctaatattattggtctcagttcaattttcgatggagtggcgtacaagtgatccagaaagcaagaatcatggatttcctacaatttcatttagattttcgatgttggttgagttatgctgattgatttgggaaagagggagcttagcgttgtatacagggttcaaacaccgtaatatgaagaggggtgtgagaccaccgggtgtgggatgtacagggcggcaaagaaacaatctaatcatagtggcaatcatatgtttggtttttatagcgatattcatcccaccgtttcttgaatgaattccataacgaagacaccgtctaaagcttcacaggttagtgcagaaatgattggttcgccctcgctatgccagtcaggcttactgagttctacttggatcgttctacttggatcttttatggcttcctagcagtcggaggtttctttctggtttgaagaaagccatgtatggaacgtttacaggttttggagaagaaagtatcaagctatttgaaaaaagtcactctggaaacttacagtaaagaggaacgccgtagtccagggaacacaacaggtgacattgtttcgctggaagatgtgatagatcgcgcctggtctgccggcgccaaagcttgggaagagctggaaattgcattcagacagggagaacatttttcgaagaaggacaataatgccaatgcaactgcagatccatgcccagcatcactctttacaacaggaaaggaattggacaatttaggaagggtcttcccactgccttgtggtctaatgtttggatcagccataactctcattggaaagccacgggaagctcacatggagtacaaaccgccaatcgccagagttggggaaggtgtctctccatacgtcatggtgtcccagttcataatggagttacagggcttgaaggtggtaaaaggtgaagatcctcctagaatcctccacataaaccctcgactccgtggtgactggagctggaaacccatcattgagcataatacatgctatcgaaaccagtggggcccagctcatcggtgtgaaggttggcaagtacctgaatacgaagaaaccggtgagtgctggttccatcacactttatcttttcatagtgacacggttctttttaggtgtactagtgttgaaagctgtgcatgttaaatggtaaccctaatcaatcttctcgctaattttcgcattgcaaggtctccgctgcttg

SEQ ID NO: 19 - protein, 634 aa, Physcomitrella patens
MKRGSRLPDMACTGRQRNDLILVAIVCLFFMVIFIPPYLQMNSLPDIDSPDSDKKSSSYSKKTTLEANSKEERRSPGNTTGDIVSLDDVIDRAWSAGAKAWEELETALRNGEGVSKNVSNATANADPCPASLSAAGKKLDELGKVFPLPCGLMFGSAITLIGKPREAHMEYKPPIARVGEGVSPYVMVSQFLVELQGLKVVKGEDPPRILHLNPRLRGDWSWKPIIEHNTCYRNQWGPAHRCEGWQVPEYEETVDGLPKCEKWLRDDGKKPASTQKSWWLGRLVGRSDKETLEWEYPLSEGREFVLTIRAGVEGFHVTIDGRHISSFPYRVGYAVEETTGILVAGDVDVMSITVTSLPLTHPSYYPELVLESGDIWKAPPVPATKIDLFIGIMSSSNHFAERMAVRKTWFQSKAIQSSQAVARFFVALHANKDINMQLKKEADYYGDIIILPFIDRYDIVVLKTVEICKFGVQNVTAKYIMKCDDDTFVRIDSVLEEIRTTSISQGLYMGSMNEFHRPLRSGKWAVTAEEWPERIYPIYANGPGYILSEDIVHFIVEMNERGSLQLFKMEDVSVGIWVREYAKQVKHVQYEHSIRFAQAGCIPKYLTAHYQSPRQMLCLWDKVLAHDDGKCCNL

SEQ ID NO: 20 - protein, 633 aa, Physcomitrella patens
MKRGVRPPGVGCTGRQRNNLIIVAIICLVFIAIFIPPFLEMNSLPDIDSPVLEKKVSSYLKKVTLETYSKEERRSPGNTTGDIVSLEDVIDRAWSAGAKAWEELEIAFRQGEHFSKKDNNANATADPCPASLFTTGKELDNLGRVFPLPCGLMFGSAITLIGKPREAHMEYKPPIARVGEGVSPYVMVSQFIMELQGLKVVKGEDPPRILHINPRLRGDWSWKPIIEHNTCYRNQWGPAHRCEGWQVPEYEETVDGLPKCEKWLRGDDKKPASTQKSWWLGRLVGHSDKETLEWEYPLSEGREFVLTIRAGVEGFHLTIDGRHISSFPYRAGYAMEEATGISVAGDVDVLSMTVTSLPLTHPSYYPELVLDSGDIWKAPPLPTGKIELFVGIMSSSNHFAERMAVRKTWFQSLVIQSSQAVARFFVALHANKDINLQLKKEADYYGDMIILPFIDRYDIVVLKTVEIFKFGVQNVTVSHVMKCDDDTFVRIDSVLEEIRTTSVGQGLYMGSMNEFHRPLRSGKWAVTVEEWPERIYPTYANGPGYILSEDIVHFIVEESKRNNLRLFKMEDVSVGIWVREYAKMKYVQYEHSVRFAQAGCIPNYLTAHYQSPRQMLCLWDKVLATNDGKCCTL

SEQ ID NO: 21 - protein, 422 aa, homo sapiens
MLQWRRRHCCFAKMTWNAKRSLFRTHLIGVLSLVFLFAMFLFFNHHDWLPGRAGFKENPVTYTFRGFRSTKSETNHSSLRNIWKETVPQTLRPQTATNSNNTDLSPQGVTGLENTLSANGSIYNEKGTGHPNSYHFKYIINEPEKCQEKSPFLILLIAAEPGQIEARRAIRQTWGNESLAPGIQITRIFLLGLSIKLNGYLQRAILEESRQYHDIIQQEYLDTYYNLTIKTLMGMNWVATYCPHIPYVMKTDSDMFVNTEYLINKLLKPDLPPRHNYFTGYLMRGYAPNRNKDSKWYMPPDLYPSERYPVFCSGTGYVFSGDLAEKIFKVSLGIRRLHLEDVYVGICLAKLRIDPVPPPNEFVFNHWRVSYSSCKYSHLITSHQFQPSELIKYWNHLQQNKHNACANAAKEKAGRYRHRKLH

SEQ ID NO: 22 - protein, 621 aa, Oryza sativa
MWVTKRLGITVLIVLFPLLIVHHLIVNSPVSGPSRYQVIHSNLLGWLSDSLGNSVAQNPDNTPVEVIPADASASNSSDSGNSSLEGFQWLNTWNHMKQLTNISDGLPHANEAIDNARTAWENLTISVHNSTSKQIKKERQCPYSIHRMNASKPDTGDFTIDIPCGLIVGSSVTIIGTPGSLSGNFRIDLVGTELPGGSGKPIVLHYDVRLTSDELTGGPVIVQNAFTASNGWGYEDRCPCSNCNNATQVDDLERCNSMVGREEKRAINSKQHLNAKKDEHPSTYFPFKQGHLAISTLRIGLEGIHMTVDGKHVTSFPYKAGLEAWFVTEVGVSGDFKLVSAIASGLPTSEDLENSFDLAMLKSSPIPEGKDVDLLIGIFSTANNFKRRMAIRRTWMQYDAVREGAVVVRFFVGLHTNLIVNKELWNEARTYGDIQVLPFVDYYSLITWKTLAICIYGTGAVSAKYLMKTDDDAFVRVDEIHSSVKQLNVSHGLLYGRINSDSGPHRNPESKWYISPEEWPEEKYPPWAHGPGYVVSQDIAKEINSWYETSHLKMFKLEDVAMGIWIAEMKKGGLPVQYKTDERINSDGCNDGCIVAHYQEPRHMLCMWEKLLRTNQATCCN

SEQ ID NO: 23 - protein, 643 aa, Arabidopsis thaliana
MKRFYGGLLVVSMCMFLTVYRYVDLNTPVEKPYITAAASVVVTPNTTLPMEWLRITLPDFMKEARNTQEAISGDDIAVVSGLFVEQNVSKEEREPLLTWNRLESLVDNAQSLVNGVDAIKEAGIVWESLVSAVEAKKLVDVNENQTRKGKEELCPQFLSKMNATEADGSSLKLQIPCGLTQGSSITVIGIPDGLVGSFRIDLTGQPLPGEPDPPIIVHYNVRLLGDKSTEDPVIVQNSWTASQDWGAEERCPKFDPDMNKKVDDLDECNKMVGGEINRTSSTSLQSNTSRGVPVAREASKHEKYFPFKQGFLSVATLRVGTEGMQMTVDGKHITSFAFRDTLEPWLVSEIRITGDFRLISILASGLPTSEESEHVVDLEALKSPTLSPLRPLDLVIGVFSTANNFKRRMAVRRTWMQYDDVRSGRVAVRFFVGLHKSPLVNLELWNEARTYGDVQLMPFVDYYSLISWKTLAICIFGTEVDSAKFIMKTDDDAFVRVDEVLLSLSMTNNTRGLIYGLINSDSQPIRNPDSKWYISYEEWPEEKYPPWAHGPGYIVSRDIAESVGKLFKEGNLKMFKLEDVAMGIWIAELTKHGLEPHYENDGRIISDGCKDGYVVAHYQSPAEMTCLWRKYQETKRSLCCREW

SEQ ID NO: 24 - DNA, 2387 bp, Physcomitrella patens
atgcgaggaggaggctgtgtttgttgcccgaagagatgggatggtttatgtgtagtgcaggggttggatgtgaagcacctgtttgaaggagtctgcgagagtttgaaattcggattcagagtgcggcgatcgatggtgcaacgttgttagcagtgattgttttcgccaacagaactgacatcatttggattttttttacgcgtggatgtgccctctttttaaaaaatttccgcgtggaaaagagacgggggtttgtaatggaggcaggctgtggtcatcacccctagtatagcctgtcaagagagttcaaattcggtaatatgaagagggggtcgagactaccggatatggcgtgtacagggcggcaaagaaatgatcttatcctagttgcaattgtttgcttgttttttatggtgatattcatcccaccatatctccaaatgaactcacttccggacattgattctcctgtcgagaagctagaagatgatgatgatgctgtcttcacttctcatagacgtcgtaaccaagagcagatttcagttgtcactgacagtggtcagagacggacagttatgccatcttcgactggtgcggaggacgtaacgaatgcaccgtctaaagattcacaggattcggacaagaaatcatcaagctactcgaaaaaaaccactctagaagccaatagtaaggaggaacgccgtagtccggggaataccacaggcgacattgtttctctggatgatgtgatagatcgtgcctggtctgctggtgccaaagcgtgggaagaactggaaactgcgttaagaaatggagaaggtgtctcaaagaatgtcagtaatgccactgcaaatgctgatccgtgtccagcatcactctctgcagcagggaaaaagttagacgaattgggtaaagtcttccccttgccctgtggtctaatgtttgggtcagccattactctgattggaaagcctcgagaggctcacatggagtacaaaccgccaatcgccagagttggggaaggcgtctctccatatgtcatggtttcccagttcttagtagagttacaaggcttaaaggtggtgaaaggtgaagatcctcctcgaattctacacttgaatcctcgacttcgtggtgattggagctggaaacccatcattgagcacaacacttgttatcggaaccagtggggtcctgcccaccgatgcgagggttggcaagtgcctgaatacgaagaaactgttgacggtcttcccaagtgcgagaagtggcttcgagatgatggcaagaaacctgcttcaacgcaaaaatcttggtggcttggaagattagttggtcgttctgacaaggagacgcttgaatgggagtacccattatctgagggtcgggagttcgttctcaccattcgagcaggtgttgaagggtttcatgtgactatcgatggtcgtcacatcagctcgtttccttatcgtgtgggttacgctgtggaagaaacaacggggatattagtagcaggagacgttgatgtgatgtctatcacagtgacatccctacccttaacacatcctagctactaccctgagttagttttggaatcgggggacatttggaaggcaccacctgtcccagctaccaagatagatttatttattgggatcatgtccagcagtaaccattttgcagaacggatggcagtaaggaagacgtggtttcaatctaaagctattcaatcttcgcaggccgtggctcgcttctttgtagctctgcatgcaaacaaggatatcaatatgcagttgaagaaggaggcagactattatggcgatattataatcctgcctttcatcgacagatatgatatagtggttctcaagaccgttgaaatttgcaagtttggggtccagaatgtcacagctaagtatattatgaagtgtgacgatgacacttttgtgaggattgatagcgttctcgaagagattcgaactacttcaatatcacaaggcctttacatgggtagcatgaatgagtttcacaggcctcttcgttctggaaagtgggccgtgactgccgaggaatggcctgagcgaatttacccaatatatgctaatggaccaggatatatcctgtcagaggatattgtgcatttcattgtggagatgaatgagagaggcagtttgcagttatttaagatggaggacgtcagtgttggaatatgggtacgcgaatatgcgaagcaagtgaagcacgttcaatacgaacatagcatacggtttgctcaagccggttgtataccgaaatacttgacagctcattaccaatcgccgcgtcaaatgctgtgtctgtgggacaaggtacttgctcatgacgatgggaaatgctgcaacttgtga

SEQ ID NO: 25 - DNA, 2052 bp, Physcomitrella patens
atgaagaggggtgtgagaccaccgggtgtgggatgtacagggcggcaaagaaacaatctaatcatagtggcaatcatatgtttggtttttatagcgatattcatcccaccgtttcttgaaatgaattcacttcccgatattgattcccctgtgtataggttagaaggtattaacttcgcttcacatagacgtcgctatcaagaacaggattcacgtgtcagttacagtggctatggacagccagatatgccatcaactggtgatgaagacataacgaagacaccgtctaaagcttcacaggttttggagaagaaagtatcaagctatttgaaaaaagtcactctggaaacttacagtaaagaggaacgccgtagtccagggaacacaacaggtgacattgtttcgctggaagatgtgatagatcgcgcctggtctgccggcgccaaagcttgggaagagctggaaattgcattcagacagggagaacatttttcgaagaaggacaataatgccaatgcaactgcagatccatgcccagcatcactctttacaacaggaaaggaattggacaatttaggaagggtcttcccactgccttgtggtctaatgtttggatcagccataactctcattggaaagccacgggaagctcacatggagtacaaaccgccaatcgccagagttggggaaggtgtctctccatacgtcatggtgtcccagttcataatggagttacagggcttgaaggtggtaaaaggtgaagatcctcctagaatcctccacataaaccctcgactccgtggtgactggagctggaaacccatcattgagcataatacatgctatcgaaaccagtggggcccagctcatcggtgtgaaggttggcaagtacctgaatacgaagaaaccgtggacggtcttcccaagtgcgagaagtggcttcgaggcgatgacaaaaaacctgcttcgacccaaaaatcctggtggcttgggcgattagttggtcattccgacaaggagacgcttgaatgggagtatccattgtccgaaggtcgggagtttgttctcaccattcgagcaggtgtagaaggatttcacttaactattgatggtcggcacatcagttcgttcccttatcgtgcgggttatgctatggaagaagcaacaggaatatcagtggcaggagacgtcgatgttctttcgatgacagtaacatcattacctttaacacatcccagctactaccctgagttggttttggattcgggtgatatctggaaggcaccacctttaccaacaggcaagatagagttatttgttggaatcatgtcaagcagcaatcactttgcagaacgtatggcagtaagaaagacgtggtttcagtctctggttatccaatcctcccaagcggtggctcgcttctttgtagctctgcatgcaaacaaggatatcaatctgcagctgaagaaagaggctgactattacggcgatatgataattttacctttcatcgacagatatgatatagtggttcttaagaccgttgaaattttcaagtttggggtccagaatgttacagttagccacgtcatgaaatgtgacgatgacacatttgtaaggattgacagcgttcttgaagagattcgaacgacgtcagtaggacagggcctttacatgggcagcatgaatgagtttcatagaccccttcgttctgggaagtgggccgtgacagttgaggagtggcctgagcgcatttacccaacatacgcaaatggtccaggatacatcctttcggaagatattgtgcattttatagtggaggagagcaaaagaaataatttgaggttatttaagatggaggacgtcagcgtaggtatatgggtacgcgagtatgcaaagatgaagtacgtgcaatacgagcatagcgtacggtttgctcaagccggttgtatacctaactacctgacagcgcactatcaatcgccgcgtcaaatgctgtgtctgtgggacaaggtgcttgctaccaatgacggcaagtgctgcaccttgtga

SEQ ID NO: 26 - protein, 688 aa, Physcomitrella patens
MKRGSRLPDMACTGRQRNDLILVAIVCLFFMVIFIPPYLQMNSLPDIDSPVEKLEDDDDAVFTSHRRRNQEQISVVTDSGQRRTVMPSSTGAEDVTNAPSKDSQDSDKKSSSYSKKTTLEANSKEERRSPGNTTGDIVSLDDVIDRAWSAGAKAWEELETALRNGEGVSKNVSNATANADPCPASLSAAGKKLDELGKVFPLPCGLMFGSAITLIGKPREAHMEYKPPIARVGEGVSPYVMVSQFLVELQGLKVVKGEDPPRILHLNPRLRGDWSWKPIIEHNTCYRNQWGPAHRCEGWQVPEYEETVDGLPKCEKWLRDDGKKPASTQKSWWLGRLVGRSDKETLEWEYPLSEGREFVLTIRAGVEGFHVTIDGRHISSFPYRVGYAVEETTGILVAGDVDVMSITVTSLPLTHPSYYPELVLESGDIWKAPPVPATKIDLFIGIMSSSNHFAERMAVRKTWFQSKAIQSSQAVARFFVALHANKDINMQLKKEADYYGDIIILPFIDRYDIVVLKTVEICKFGVQNVTAKYIMKCDDDTFVRIDSVLEEIRTTSISQGLYMGSMNEFHRPLRSGKWAVTAEEWPERIYPIYANGPGYILSEDIVHFIVEMNERGSLQLFKMEDVSVGIWVREYAKQVKHVQYEHSIRFAQAGCIPKYLTAHYQSPRQMLCLWDKVLAHDDGKCCNL

SEQ ID NO: 27 - protein, 683 aa, Physcomitrella patens
MKRGVRPPGVGCTGRQRNNLIIVAIICLVFIAIFIPPFLEMNSLPDIDSPVYRLEGINFASHRRRYQEQDSRVSYSGYGQPDMPSTGDEDITKTPSKASQVLEKKVSSYLKKVTLETYSKEERRSPGNTTGDIVSLEDVIDRAWSAGAKAWEELEIAFRQGEHFSKKDNNANATADPCPASLFTTGKELDNLGRVFPLPCGLMFGSAITLIGKPREAHMEYKPPIARVGEGVSPYVMVSQFIMELQGLKVVKGEDPPRILHINPRLRGDWSWKPIIEHNTCYRNQWGPAHRCEGWQVPEYEETVDGLPKCEKWLRGDDKKPASTQKSWWLGRLVGHSDKETLEWEYPLSEGREFVLTIRAGVEGFHLTIDGRHISSFPYRAGYAMEEATGISVAGDVDVLSMTVTSLPLTHPSYYPELVLDSGDIWKAPPLPTGKIELFVGIMSSSNHFAERMAVRKTWFQSLVIQSSQAVARFFVALHANKDINLQLKKEADYYGDMIILPFIDRYDIVVLKTVEIFKFGVQNVTVSHVMKCDDDTFVRIDSVLEEIRTTSVGQGLYMGSMNEFHRPLRSGKWAVTVEEWPERIYPTYANGPGYILSEDIVHFIVEESKRNNLRLFKMEDVSVGIWVREYAKMKYVQYEHSVRFAQAGCIPNYLTAHYQSPRQMLCLWDKVLATNDGKCCTL

SEQ ID NO: 28 - protein, 6 aa, Artificial Sequence
  Synthetic peptide
DLFIGI

SEQ ID NO: 29 - protein, 6 aa, Artificial Sequence
  Synthetic peptide
ELFVGI

SEQ ID NO: 30 - protein, 8 aa, Artificial Sequence
  Synthetic peptide
RMAVRKTW

SEQ ID NO: 31 - protein, 4 aa, Artificial Sequence
  Synthetic peptide
FVAL

SEQ ID NO: 32 - protein, 11 aa, Artificial Sequence
  Synthetic peptide
DRYDIVVLKTV

SEQ ID NO: 33 - protein, 11 aa, Artificial Sequence
  Synthetic peptide
YIMKCDDDTFV

SEQ ID NO: 34 - protein, 11 aa, Artificial Sequence
  Synthetic peptide
HVMKCDDDTFV

SEQ ID NO: 35 - protein, 13 aa, Artificial Sequence
  Synthetic peptide
YPIYANGPGYILS

SEQ ID NO: 36 - protein, 13 aa, Artificial Sequence
  Synthetic peptide
YPTYANGPGYILS

SEQ ID NO: 37 - protein, 7 aa, Artificial Sequence
  Synthetic peptide
EDVSVGI

(19) United States
(12) Patent Application Publication
(10) Pub. No.: US 2010/0050292 A1
(43) Pub. Date: Feb. 25, 2010

(54) Galactosyltransferase

(75) Inventors: Heike Launhardt, Freiburg (DE); Christian Stemmer, Freiburg (DE); Wolfgang Jost, Freiburg (DE); Gilbert Gorr, Freiburg (DE); Ralf Reski, Oberried (DE); Stefan Rensing, Gundelfingen (DE)

Correspondence Address: FULBRIGHT & JAWORSKI L.L.P., 600 CONGRESS AVE., SUITE 2400, AUSTIN, TX, 78701, US

(73) Assignee: GREENOVATION BIOTECH GMBH, Freiburg (DE)

(21) Appl. No.: 12/443,377

(22) PCT Filed: Sep. 28, 2007
(86) PCT No.: PCT/EP2007/008465

§ 371 Date: Mar. 27, 2009

(30) Foreign Application Priority Data
Sep. 29, 2006 (EP) 06450139.8

(51) Int. Cl. A01H 1/00; C07H 21/04; C12N 15/63; C12P 21/06; C12N 9/10; C12N 15/82; C07K 14/00; C12Q 1/68

(52) U.S. Cl. 800/278; 536/23.2; 435/320.1; 435/69.1; 435/193; 435/468; 530/395; 435/6

(57) ABSTRACT

The invention discloses DNA molecules encoding galactosyltransferases, recombinant host cells, tissues or organisms comprising dysfunctional galactosyltransferase gene(s), recombinant host cells, tissues or organisms comprising an introduced functional galactosyltransferase gene, methods for the production of proteins therewith, methods for the production of galactosyltransferase and vectors and uses thereof.

The present invention relates to polynucleotides coding for glycosyltransferases. Moreover, the present invention relates to partial polynucleotides thereof as well as to vectors comprising these polynucleotides in purposes of expression or gene disruption thereof, recombinant host cells, tissue or organisms transfected with the polynucleotides or parts thereof or DNA derived therefrom, as well as glycoproteins produced in these host cells, tissue or organisms. Furthermore, the present invention relates to the use of the expression product thereof in vitro as well as in vivo.

In the past, heterologous proteins have been produced using a variety of transformed cell systems, such as those derived from bacteria, fungi, such as yeasts, insect, plant or mammalian cell lines.

Proteins produced in prokaryotic organisms may not be post-translationally modified in a similar manner to that of eukaryotic proteins produced in eukaryotic systems, e.g. they may not be glycosylated with appropriate sugars at particular amino acid residues, such as aspartic acid (N) residues (N-linked glycosylation). Furthermore, folding of bacterially-produced eukaryotic proteins may be inappropriate due to, for example, the inability of the bacterium to form cysteine disulfide bridges. Moreover, bacterially-produced recombinant proteins frequently aggregate and accumulate as insoluble inclusion bodies.

Eukaryotic cell systems are better suited for the production of glycosylated proteins found in various eukaryotic organisms, such as humans, since such cell systems may effect post-translational modifications, such as N-glycosylation of produced proteins. However, a problem encountered in eukaryotic cell systems which have been transformed with heterologous genes suitable for the production of protein sequences destined for use, for example, as pharmaceuticals, is that the glycosylation pattern on such proteins often acquires a native pattern, that is, of the eukaryotic cell system in which the protein has been produced: glycosylated proteins are produced that comprise non-animal glycosylation patterns and these in turn may be immunogenic and/or allergenic if applied in animals, including humans. In plants this limitation has been overcome by the elimination of the plant-specific sugar residues 1,2-xylose and α1,3-fucose which in plants are generally linked to the core structure of N-glycans (Lerouge et al. 1998 Plant Mol. Biol. 38, 31-48; Rayon et al. 1998 J. Experimental Bot. 49, 1463-1472). In case of Arabidopsis thaliana (Strasser et al. 2004 FEBS Lett. 561, 132-136) and in case of the bryophyte Physcomitrella patens (EP1431394 Koprivova et al. 2004 Plant Biotechnol. J. 2, 517-523) mutants were generated showing N-glycan patterns completely lacking core α1,3-fucose and 1,2-xylose residues. Surprisingly, despite the modification of the pattern of the complex-type N-glycans no morphological alterations or changes in viability were observed in these mutants.

Apart from the addition of the two plant-specific residues described above the steps of glycoprotein maturation in the ER and in the cis-Golgi are identical in plants and mammals up to the action of GlcNAc-transferase I, GlcNAc-transferase II and Golgi a-mannosidase (Lerouge et al. 1998 Plant Mol. Biol. 38, 31-48). Further N-glycan elongation is carried out in a different manner in the two kingdoms. While in mammals the terminal GlcNAc residues are immediately shielded by the action of β1,4- (or, seldom, by β1,3-)-galactosyltransferase—with the notable exception of IgG where this step only occurs partially—elongation in plants is exclusively by β1,3-galactosylation but only a very small part of the glycans appear to undergo this modification as can be deduced from the relative abundance of various structural types. The galactose-residues in mammals may be capped by sialic acid and only quite rarely substituted by fucose. Again, plants are different, as they are devoid of sialylation and in case that a terminal 1,3-linked galactose residue was attached they essentially always fucosylate the pen-ultimate GlcNAc residue, thereby forming a Lewis a (LeA) determinant. Apparently, the β1,3-galactosyltransferase is the limiting enzyme whereas most plant cells contain sufficient activity of α1,4-Fuc-transferase to make sure that each Gal containing antenna is fucosylated. The LeA structure is a human blood group determinant. It is rare as such in healthy adults but as sialyl-Lewis a (sLeA) it is notoriously found in malignant tissues such as colon cancer.

Anyway, LeA containing glycoproteins are rarely isolated from plants and in case of Physcomitrella they present an amount of only up to five percent of totally soluble glyco-proteins—irrespective if isolated from wild type plants or isolated from the glyco-engineered mutants lacking core fucose and xylose (Koprivova et al. 2003 Plant Biol. 5, 582-591; Koprivova et al. 2004 Plant Biotechnol. J. 2, 517-523).

Whereas some investigations were performed regarding the α1,4-fucosyltransferase which is involved in the generation of Lewis a type glycan structures in plants (Joly et al. 2002 J. Experimental Bot. 53, 1429-1436; Bakker et al. 2001 FEBS Lett. 507, 307-312) there is no information available regarding a specific β1,3 galactosyltransferase which is involved in the elongation of N-glycan structures in plants.

In eukaryotes β1,3-galactosyltransferases show a broad spectrum of acceptor specifities as well as distinct patterns of tissue expression (Hennet 2002 Cell. Mol. Life Sci. 59, 1081-1095; Amado et al. 1998 J. Biol. Chem. 21, 12770-12778). Among the different members of the β1,3-galactosyltransferase family of humans for β1,3-galactosyltransferase 2 it has been shown in vitro that this enzyme was active toward the transfer of galactose residues to GlcNAcβand egg ovalbumin—representing complex-type N-glycan structures as acceptor substrates (Amado et al. 1998 J. Biol. Chem. 21, 12770-12778).

According to the existence of a family of homologous β1,3-galactosyltransferases in humans data base analysis revealed that in different plant species e.g. Arabidopsis thaliana and Oryza sativa similar large gene families of β1,3-galactosyltransferase genes exist. None of the members of these β1,3-galactosyltransferase genes is described as coding for an enzyme which comprise the ability to transfer galactose from UDP-galactose to acceptor substrates with terminal non-reducing GlcNAc residues e.g. to non-reducing terminal residues of the complex-type N-glycans neither in vitro nor in vivo.

It is an object of the present invention to identify and to clone and to sequence one or more genes—including non-coding corresponding genomic sequences—which code for plant β1,3galactosyltransferases, and to prepare vectors comprising the genes, DNA fragments thereof or an altered DNA or a DNA derived thereof or DNA comprising deletions thereof. It is a further objective to generate host cells, tissue or organisms comprising one or more of these vectors, to produce glycoproteins completely lacking Lewis a type N-glycan structures. It is a further objective to generate host cells, tissue or organisms comprising one or more of these vectors, to produce glycoproteins with improved Lewis a type N-glycan structures. It is a further objective to provide nucleotide sequences encoding membrane domains for targeting enzymes to the late Golgi cisternae.

Accordingly, the present invention provides

i) a DNA molecule comprising a sequence according to SEQ ID NO: 1 having an open reading frame from base pair 513 to base pair 2417 or having at least 50% identity with the above-mentioned sequence or comprising a sequence which has degenerated to the above DNA sequence due to the genetic code, the sequence coding for a plant protein which has β1,3-galactosyltransferase activity or is complementary thereto, ii) a DNA molecule comprising a sequence according to SEQ ID NO: 2 having an open reading frame from base pair 1 to base pair 1902 or having at least 50% identity with the above-mentioned sequence or comprising a sequence which has degenerated to the above DNA sequence due to the genetic code, the sequence coding for a plant protein which has β1,3-galactosyltransferase activity or is complementary thereto, iii) a DNA molecule comprising a sequence according to SEQ ID NO: 24 having an open reading frame from base pair 321 to base pair 2387 or having at least 50% identity with the above-mentioned sequence or comprising a sequence which has degenerated to the above DNA sequence due to the genetic code, the sequence coding for a plant protein which has β1,3-galactosyltransferase activity or is complementary thereto, iv) a DNA molecule comprising a sequence according to SEQ ID NO: 25 having an open reading frame from base pair 1 to base pair 2052 or having at least 50% identity with the above-mentioned sequence or comprising a sequence which has degenerated to the above DNA sequence due to the genetic code, the sequence coding for a plant protein which has β1,3-galactosyltransferase activity or is complementary thereto, v) a DNA molecule comprising a sequence according to SEQ ID NO: 3 representing the genomic DNA structure from base pair 1 to base pair 6187 including intron sequences and exon sequences corresponding to SEQ ID NO: 1 allowing generation of knockout constructs with genomic sequences, vi) a DNA molecule comprising a sequence according to SEQ ID NO: 4 representing the genomic DNA structure from base pair 1 to base pair 4087 including intron sequences and exon sequences corresponding to SEQ ID NO: 2 allowing generation of knockout constructs with genomic sequences.

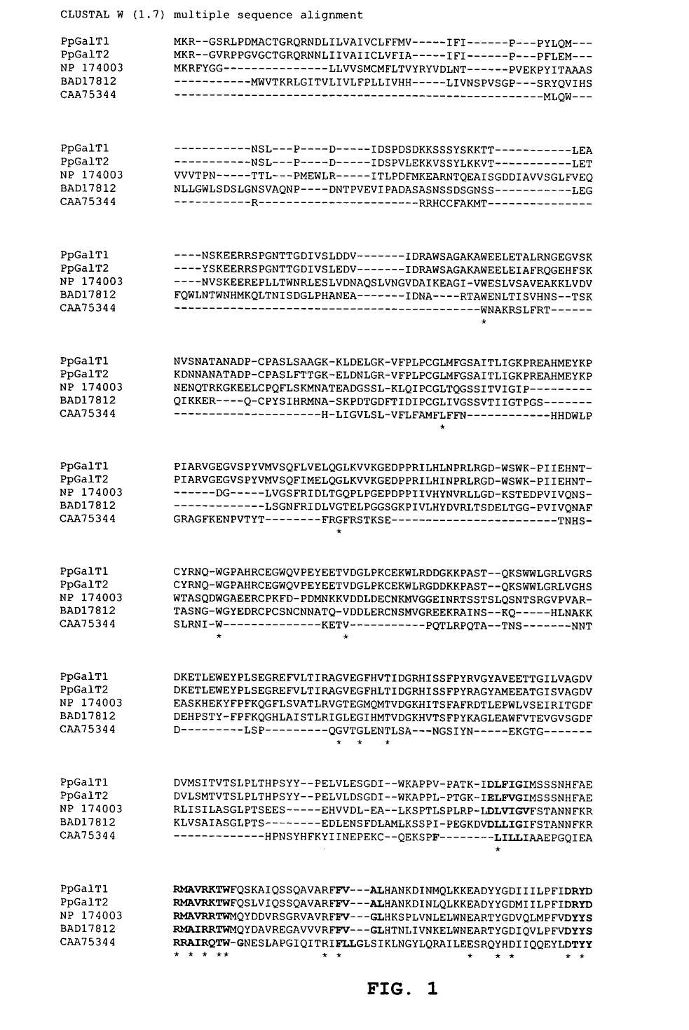

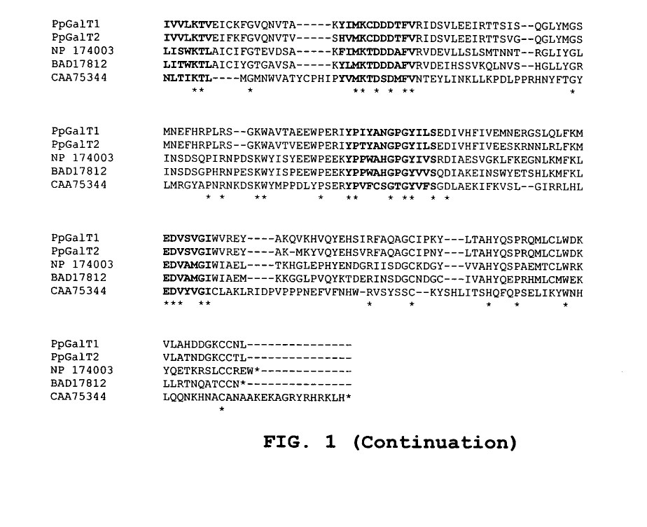

Since the family of glycosyltransferases is highly divergent (FIG. 1) and only conserved regions (bold in FIG. 1) are highly similar, the present invention also provides a DNA molecule comprising a sequence having at least 20% overall identity to a sequence according to any one of SEQ ID NO: 1, SEQ ID NO: 2, SEQ ID NO: 24 or SEQ ID NO: 25 and having at least 80% identity to a sequence of the seven conserved domains of SEQ ID NO: 1 or SEQ ID NO: 2 encoding amino acids 387-392 (DLFIGI or ELFVGI), 402-409 (RMAVRKTW), 425-428 (FVAL), 455-465 (DRYDIVVLKTV), 479-489 (YIMKCDDDTFV or HVMKCDDDTFV), 536-548 (YPIYANGPGYILS or YPTYANGPGYILS) and 570-576 (EDVSVGI) of the protein of SEQ ID NO: 19 or SEQ ID NO: 20, or comprising a sequence which is degenerated to the above sequence due to the genetic code, with the sequence coding for plant proteins having β1,3-galactosyltransferase activity or being complementary thereto. Also provided is the DNA molecule comprising a sequence having at least 20% overall identity to a sequence according to any one of SEQ ID NO: 1, SEQ ID NO: 2, SEQ ID NO: 24 or SEQ ID NO: 25 and encoding at least 95%, preferably all, of the conserved amino acids of the seven conserved domains of SEQ ID NO: 1 or SEQ ID NO: 2 selected from amino acids 388 (L), 402 (R), 404 (A), 406 (R), 408 (T), 409 (W), 425 (F), 455 (D), 457 (Y), 463 (K), 464 (T), 481 (M), 482 (K), 484 (D), 486 (D), 488 (F), 489 (V), 536 (Y), 537 (P), 542 (G), 544 (G), 545 (Y), 548 (S), 570 (E), 571 (D), 572 (V), 575 (G) and 576 (I) of the protein of SEQ ID NO: 19 or SEQ ID NO: 20, or comprising a sequence which is degenerated to the above sequence due to the genetic code, with the sequence coding for plant proteins having β1,3-galactosyltransferase activity or being complementary thereto. Preferably the overall sequence identity is at least 25%, at least 30%, at least 35%, at least 40% or at least 45%. In further preferred embodiments the sequence identity for the conserved domains is at least 90%, at least 95% or 100%.

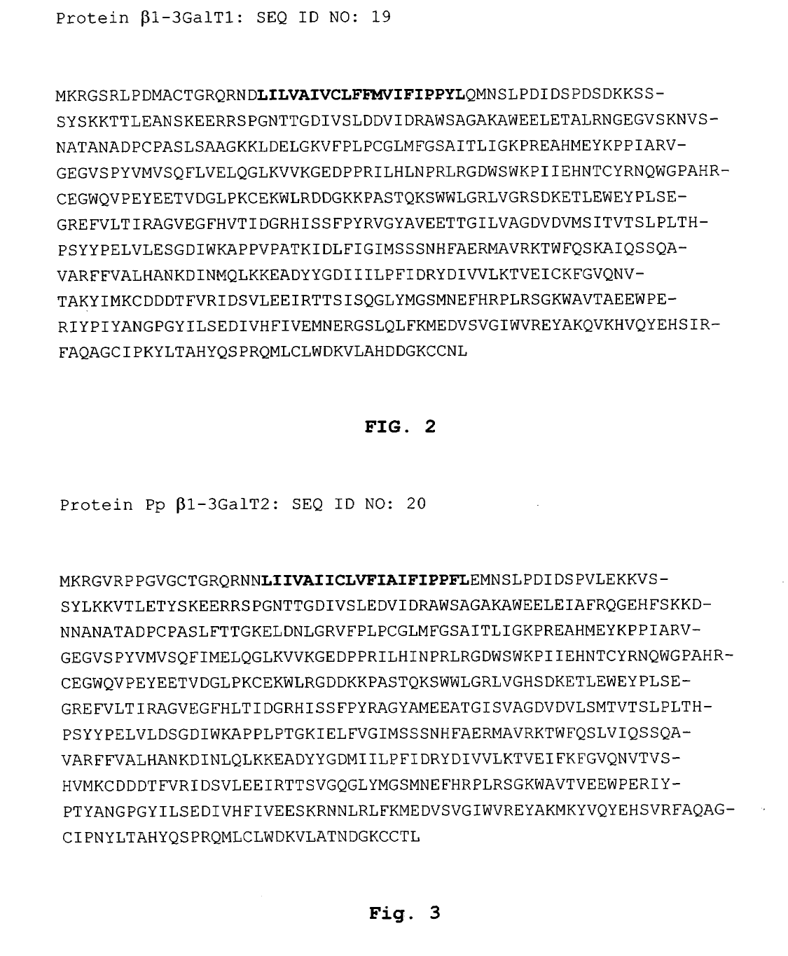

The open reading frame of the sequence having SEQ ID NO: 1 codes for a protein with 634 amino acids (FIG. 2, SEQ ID NO: 19). The protein encoded by SEQ ID NO: 1 contains a transmembrane domain in the region between Leu20 and Leu39, and encloses the seven conserved domains—present in human β1,3-galactosyltransferases—described by Hennet (2002 Cell. Mol. Life Sci. 59, 1081-1095, FIG. 2) as well as most of the C-terminal located conserved amino acids as described by Amado et al. (1998 J. Biol. Chem. 21, 12770-12778, FIG. 2).

The open reading frame of the sequence having SEQ ID NO: 2 codes for a protein with 633 amino acids (FIG. 3, SEQ ID NO: 20). The protein encoded by SEQ ID NO: 2 contains a transmembrane domain in the region between Leu20 and Leu39, and encloses the seven conserved domains—present in human β1,3-galactosyltransferases—described by Hennet (2002 Cell. Mol. Life Sci. 59, 1081-1095, FIG. 2) as well as most of the C-terminal located conserved amino acids as described by Amado et al. (1998 J. Biol. Chem. 21, 12770-12778, FIG. 2).

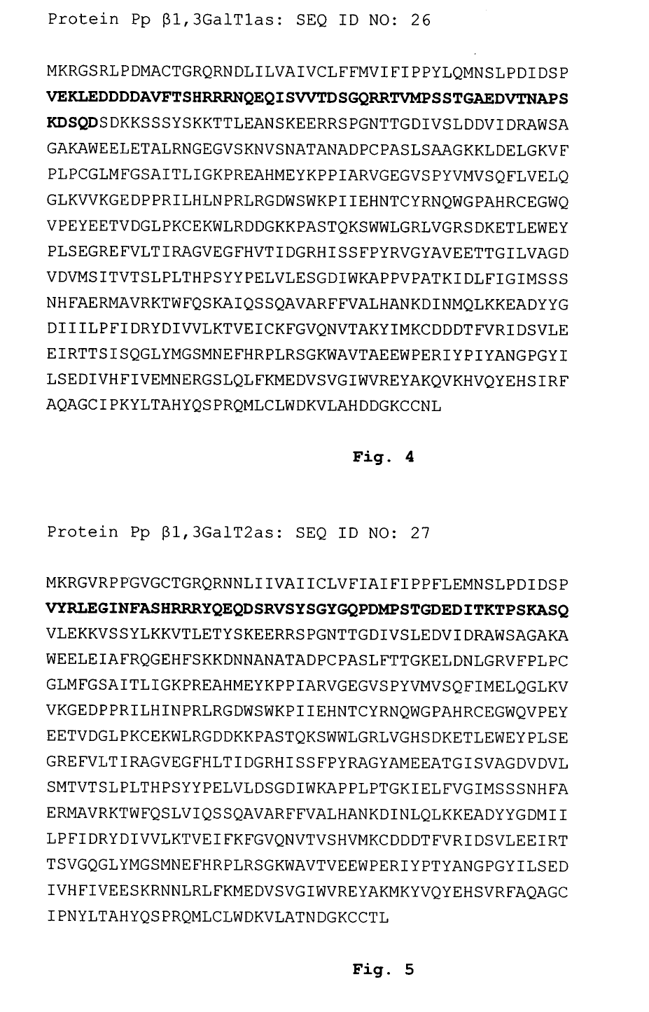

The open reading frame of the sequence having SEQ ID NO: 24 codes for a protein with 688 amino acids (FIG. 4; SEQ ID NO: 26), which is an alternative splice variant to the protein of SEQ ID NO: 1.

The open reading frame of the sequence having SEQ ID NO: 25 codes for a protein with 683 amino acids (FIG. 5, SEQ ID NO: 27), which is an alternative splice variant to the protein of SEQ ID NO: 2.

The present invention also relates to the genomic sequences of this gene as given by SEQ ID NOs. 3 or 4, of course, as all other DNA molecules or proteins according to the present invention (if not explicitly described otherwise) in isolated form.

Activity of the plant β1,3-galactosyltransferases can be analysed by different approaches.

According to Amado et al. (1998 J. Biol. Chem. 21, 12770-12778) constructs encoding the soluble secreted forms—lacking the transmembrane domain—of the β1,3-galactosyltransferases can be cloned into expression vectors e.g. appropriate for transfection of Baculo virus and amplified in Sf9 cells; the resulting expression products can be purified and subsequently assayed for β1,3-galactosyltransferase activity.

Another approach due to the analyses of specific activity can be the overexpression of the β1,3-galactosyltransferases in an appropriate host e.g. like Physcomitrella patens by preparing expression constructs designed to encode the full open reading frames of the β1,3-galactosyltransferases according to the present invention and by generation of Physcomitrella strains transgenic for at least one of the β1,3-galactosyltransferase genes according to the present invention. The generated trans-genic strains show improved contents of galactosylated N-glycans. N-glycan patterns from Physcomitrella can be isolated and analysed as described by Koprivova et al. (2003 Plant Biol. 5, 582-591) and Koprivova et al. (2004 Plant Biotechnol. J. 2, 517-523).

β1,3-galactosyltransferase activities according to the present invention can be assayed indirectly by targeted disruption of the responsible genes in an appropriate host e.g. Physcomitrella patens which result in inhibition of β1,3-galactosyltransferase activities in respect to the transfer of galactose from UDP-galactose to the non-reducing terminal GlcNAc residues on N-glycans and therefore to the lack of terminal galactosylation. Again, N-glycan patterns from Physcomitrella can be isolated and analysed as described by Koprivova et al. (2003 Plant Biol. 5, 582-591) and Koprivova et al. (2004 Plant Biotechnol. J. 2, 517-523). Preferably, the β1,3-galactosyltransferase according to the present invention is a GlcNAc-β1,3-galactosyltransferase. Alternatively reduction of β1,3-galactosyltransferase activity can be achieved by methods which are commonly used for this kind of purpose e.g. the well known antisense strategy, sense strategy, ribozyme technology, PNA technology or RNA interference strategy.

According to the present invention a host cell, tissue or organism is transfected with the nucleotide sequences comprising at least the sequences of SEQ ID NO: 1, SEQ ID NO: 2, SEQ ID NO: 24 or SEQ ID NO: 25 which code for a functional β1,3-galactosyltransferase. In a preferred embodiment of this invention the coding sequences are linked to regulatory sequences such as promoter and termination sequences allowing expression of the β1,3-galactosyltransferase genes resulting in the expression products which show β1,3-galactosyltransferase activities. Regarding the host cell tissue or organism the regulatory sequences operably linked to the β1,3-galactosyltransferase coding sequence can be heterologous. In another embodiment the regulatory sequences operably linked to the β1,3-galactosyltransferase coding sequence can be homologous due to the used host. The regulatory sequences operably linked to the β1,3-galactosyltransferase coding sequence can be provided by the vector used for transfection or can be established in vivo by introducing the β1,3-galactosyltransferase coding sequence by targeted integration e.g. homologous recombination into an appropriate locus resulting in an operably functional assembly of the β1,3-galactosyltransferase coding sequence with the endogenous regulatory sequences of the host cell, tissue or organism.

In a preferred embodiment of the present invention the expression product or parts thereof e.g. a soluble form lacking transmembrane domains comprising β1,3-galactosyltransferase can be used for elongation of N-glycans on glycolipids or glycoproteins in vitro or in vivo. In a further embodiment the resulting N-glycans comprising terminal 1,3 linked galactose residues can be further elongated in vitro or in vivo with additional sugar residues like fucose, galactose or sialic acid residues. Accordingly, the present invention relates to novel glycoproteins with N-glycans sugar structure comprising complex type N-glycans containing terminal sugar residues, such as galactose, an additional fucose, sialic acid or combinations thereof. In a more preferred embodiment, these glycoproteins are surface proteins presenting the complex type N-glycans to the outer environment of the cell, e.g. allowing protein/protein contacts (such as contacts with antibodies, other cells, etc.) or secretory proteins, e.g. antibodies or erythropoietin. Such glycoproteins produced according to the present invention are highly suitable for vaccination, especially of humans, both in vitro and in vivo.

In another embodiment of the present invention there are provided nucleotide sequences according to SEQ ID NO: 1, SEQ ID NO: 2, SEQ ID NO: 24 or SEQ ID NO: 25 which encode transmembrane domains for targeting a heterologous protein to the late Golgi cisternae. In a preferred embodiment β1,4-galactosyltransferases or sialyltransferases showing activity for elongation of N-glycans are targeted to the late Golgi cisternae by exchange of the native transmembrane domains with these of the 1,3-galactosyltransferases according to the present invention.

According to the present invention there is provided a trans-formed host cell that comprises at least one dysfunctional β1,3-galactosyltransferase nucleotide sequence.

In a preferred embodiment of the invention the host cell is selected from plants, e.g. Lemna species, Wolffia species, rice, carrot, corn, maize and tobacco species. In a more preferred embodiment of the present invention the host cell is selected from bryophytes including mosses and liverworts, of species from the genera Physcomitrella, Funaria, Sphagnum, Ceratodon, Marchantia and Sphaerocarpos. The bryophyte cell is preferably from Physcomitrella patens.

A preferred host according to the present invention is a bryophyte, especially Physcomitrella pa tens, a haploid non-vascular land plant, can be used for the production of glyco-engineered recombinant proteins (WO 01/25456). In Physcomitrella patens as well as in other plants Lewis a type structures have been detected (Koprivova et al. 2003 Plant Biol. 5, 582-591; Koprivova et al. 2004 Plant Biotechnol. J. 2, 517-523). Although from plants no β1,3-galactosyltransferases showing specific activity in elongation of N-glycan structures have been identified Physcomitrella was chosen as a putative source for this unknown kind of glycosyltransferase.

The life cycle of mosses is dominated by photoautotrophic gametophytic generation. The life cycle is completely different to that of the higher plants wherein the sporophyte is the dominant generation and there are notably many differences to be observed between higher plants and bryophytes.

The gametophyte of bryophytes including mosses is characterised by two distinct developmental stages. The protonema which develops via apical growth, grows into a filamentous network of only two cell types (chloronemal and caulonemal cells). The second stage, called the gametophore, differentiates by caulinary growth from a simple apical system. Both stages are photoautotrophically active. Cultivation of protonema without differentiation into the more complex gametophore has been shown for suspension cultures in flasks as well as for bioreactor cultures (WO 01/25456). Cultivation of fully differentiated and photoautrophically active multicellullar tissue containing only a few cell types is not described for higher plants. The genetic stability of the moss cell system provides an important advantage over plant cell cultures.

There are some important differences between bryophytes (non-vascular plants) and higher plants (vascular plants) on the biochemical level. Sulfate assimilation in Physcomitrella patens differs significantly from that in higher plants. The key enzyme of sulfate assimilation in higher plants is adenosine 5′-phosphosulfate reductase. In Physcomitrella patens an alternative pathway via phosphoadenosine 5′-phosphosulfate reductase co-exists (Koprivova et al. (2002) J. Biol. Chem. 277, 32195-32201). This pathway has not been characterised in higher plants.

Furthermore, many members of the bryophytes, algae and fern families produce a wide range of polyunsaturated fatty acids (Dembitsky (1993) Prog. Lipid Res. 32, 281-356). For example, arachidonic acid and eicosapentaenoic acid are thought to be produced only by lower plants and not by higher plants. Some enzymes of the metabolism of polyunsaturated fatty acids, (delta 6-acyl-group desaturase) (Girke et al. (1998), Plant J, 15, 39-48) and a component of a delta 6 elongase (Zank et al. (2002) Plant J 31, 255-268), have been cloned from Physcomitrella patens. No corresponding genes have been found in higher plants. This fact appears to confirm that essential differences exist between higher plants and lower plants at the biochemical level.

Moreover, bryophytes show highly efficient homologous recombination in its nuclear DNA, a unique feature for plants, which enables directed gene disruption (Girke et al. (1998) Plant J, 15, 39-48; Strepp et al. (1998) Proc Natl Acad Sci USA 95, 4368-4373; Koprivova (2002) J. Biol. Chem. 277, 32195-32201; reviewed by Reski (1999) Planta 208, 301-309; Schaefer and Zryd (2001) Plant Phys 127, 1430-1438; Schaefer (2002) Annu. Rev. Plant Biol. 53, 477-501; Koprivova et al. 2004 Plant Biotechnol. J. 2, 517-523; Brucker et al. 2005 Planta 220, 864-874) further illustrating fundamental differences to higher plants. However, in some cases the use of this mechanism for altering glycosylation pattern has proven to be problematic, as shown herein in the examples. Disruption of N-acetylglucosaminyltransferase I (GNT1) in Physcomitrella patens resulted in the loss of the specific transcript but only in minor differences of the N-glycosylation pattern. These results were in direct contrast to the loss of Golgi-modified complex glycans in a mutant Arabidopsis thaliana plant lacking GNT1 observed by von Schaewen et al. (1993) Plant Physiol 102, 1109-1118). Thus, the knockout in Physcomitrella patens did not result in the expected modification of the N-glycosylation pattern.

Although the knockout strategy was not successful for the glycosyltransferase GNT1, regarding the disruptions of the genes coding for the β1,2-xylosyltransferase and α1,3-galactosyltransferase knockouts were performed successfully in Physcomitrella patens.

In addition integration of the human β1,4-galactosyltransferase into the genome of a double knockout Physcomitrella patens plant resulted in a mammalian-like N-linked glycosylation pattern without the plant specific fucosyl and xylosyl residues and with mammalian-like terminal 1,4 galactosyl residues. The galactosyltransferase was found to be active.

The bryophyte cell, such as a Physcomitrella patens cell, can be any cell suitable for transformation according to methods of the invention as described herein, and may be a moss protoplast cell, a cell found in protonema tissue or other cell type. Indeed, the skilled addressee will appreciate that moss plant tissue comprising populations of transformed bryophyte cells according to the invention, such as transformed protonemal tissue also forms an aspect of the present invention.

“Dysfunctional” as used herein means that the nominated transferase nucleotide sequences of β1,3-galactosyltransferase (β1,3-GalT) are substantially incapable of encoding mRNA that codes for functional β1,3-GalT proteins that are capable of modifying plant N-linked glycans with 1,3 linked terminal galactose residues. In a preferment, the dysfunctional β1,3-GalT plant transferase nucleotide sequences comprise targeted insertions of exogenous nucleotide sequences into endogenous, that is genomic, native β1,3-GalT genes comprised in the nuclear bryophyte genome (whether it is a truly native bryophyte genome, that is in bryophyte cells that have not been transformed previously by man with other nucleic acid sequences, or in a transformed nuclear bryophyte genome in which nucleic acid sequence insertions have been made previously of desired nucleic acid sequences) which substantially inhibits or represses the transcription of mRNA coding for functional β1,3-GalT activity.

A further aspect of the invention relates to a biologically functional vector which comprises one of the above-indicated DNA molecules or parts thereof of differing lengths with at least 20 base pairs. For transfection into host cells, an independent vector capable of amplification is necessary, wherein, depending on the host cell, transfection mechanism, task and size of the DNA molecule, a suitable vector can be used. Since a large number of different vectors is known, an enumeration thereof would go beyond the limits of the present application and therefore is done without here, particularly since the vectors are very well known to the skilled artisan (as regards the vectors as well as all the techniques and terms used in this specification which are known to the skilled artisan, cf. also Sambrook Maniatis). Ideally, the vector has a small molecule mass and should comprise selectable genes so as to lead to an easily recognizable phenotype in a cell so thus enable an easy selection of vector-containing and vector-free host cells. To obtain a high yield of DNA and corresponding gene products, the vector should comprise a strong promoter, as well as an enhancer, gene amplification signals and regulator sequences. For an autonomous replication of the vector, furthermore, a replication origin is important. Polyadenylation sites are responsible for correct processing of the mRNA and splice signals for the RNA transcripts. If phages, viruses or virus particles are used as the vectors, packaging signals will control the packaging of the vector DNA. For instance, for transcription in plants, Ti plasmids are suitable, and for transcription in insect cells, baculoviruses, and in insects, respectively, transposons, such as the P element.

If the above-described inventive vector is inserted into a plant or into a plant cell, a post-transcriptional suppression of the gene expression of the endogenous β1,3galactosyltransferase gene is attained by transcription of a transgene homologous thereto or of parts thereof, in sense orientation. For this sense technique, furthermore, reference is made to the publications by Baucombe 1996, Plant. Mol. Biol., 9:373-382, and Brigneti et al., 1998, EMBO J. 17:6739-6746. This strategy of “gene silencing” is an effective way of suppressing the expression of the β1,3galactosyltransferase gene, cf. also Waterhouse et al., 1998, Proc. Natl. Acad. Sci. USA, 95:13959-13964.

Furthermore, the invention relates to a biologically functional vector comprising a DNA molecule according to one of the above-described embodiments, or parts thereof of differing lengths in reverse orientation to the promoter. If this vector is transfected in a host cell, an “antisense mRNA” will be read which is complementary to the mRNA of the β1,3galactosyltransferase and complexes the latter. This bond will either hinder correct processing, transportation, stability or, by preventing ribosome annealing, it will hinder translation and thus the normal gene expression of the β1,3galactosyltransferase.

Although the entire sequence of the DNA molecule could be inserted into the vector, partial sequences thereof because of their smaller size may be advantageous for certain purposes. With the antisense aspect, e.g., it is important that the DNA molecule is large enough to form a sufficiently large antisense mRNA which will bind to the transferase mRNA. A suitable antisense RNA molecule comprises, e.g., from 50 to 200 nucleotides since many of the known, naturally occurring antisense RNA molecules comprise approximately 100 nucleotides.

For a particularly effective inhibition of the expression of an active β1,3galactosyltransferase, a combination of the sense technique and the antisense technique is suitable (Waterhouse et al., 1998, Proc. Natl. Acad. Sci., USA, 95:13959-13964).

Advantageously, rapidly hybridizing RNA molecules are used. The efficiency of antisense RNA molecules which have a size of more than 50 nucleotides will depend on the annealing kinetics in vitro. Thus, e.g., rapidly annealing antisense RNA molecules exhibit a greater inhibition of protein expression than slowly hybridizing RNA molecules (Wagner et al., 1994, Annu. Rev. Microbiol., 48:713-742; Rittner et al., 1993, Nucl. Acids Res., 21:1381-1387). Such rapidly hybridizing antisense RNA molecules particularly comprise a large number of external bases (free ends and connecting sequences), a large number of structural subdomains (components) as well as a low degree of loops (Patzel et al. 1998; Nature Biotechnology, 16; 64-68). The hypothetical secondary structures of the antisense RNA molecule may, e.g., be determined by aid of a computer program, according to which a suitable antisense RNA DNA sequence is chosen.

Different sequence regions of the DNA molecule may be inserted into the vector. One possibility consists, e.g., in inserting into the vector only that part which is responsible for ribosome annealing. Blocking in this region of the mRNA will suffice to stop the entire translation. A particularly high efficiency of the antisense molecules also results for the 5′- and 3′-non-translated regions of the gene.

Preferably, the DNA molecule according to the invention includes a sequence which comprises a deletion, insertion and/or substitution mutation. The number of mutant nucleotides is variable and varies from a single one to several deleted, inserted or substituted nucleotides. It is also possible that the reading frame is shifted by the mutation. In such a “knock-out gene” it is merely important that the expression of a β1,3galactosyltransferase is disturbed, and the formation of an active, functional enzyme is prevented. In doing so, the site of the mutation is variable, as long as expression of an enzymatically active protein is prevented. Preferably, the mutation in the catalytic region of the enzyme which is located in the C-terminal region. The method of inserting mutations in DNA sequences are well known to the skilled artisan, and therefore the various possibilities of mutageneses need not be discussed here in detail. Coincidental mutageneses as well as, in particular, directed mutageneses, e.g. the site-directed mutagenesis, oligonucleotide-controlled mutagenesis or mutageneses by aid of restriction enzymes may be employed in this instance.

Alternatively, ribozyme or siRNA techniques may be applied for reducing or eliminating β1,3-GaltT activity in cells which have wildtype β1,3-GalT activity. Adaptation of siRNA techniques to the present invention are straight forward based on existing skills in the art (e.g. Nat. Reviews: RNA interference collection (October 2005)).

The invention further provides a DNA molecule which codes for a ribozyme which comprises two sequence portions of at least 10 to 15 base pairs each, which are complementary to sequence portions of an inventive DNA molecule as described above so that the ribozyme complexes and cleaves the mRNA which is transcribed from a natural β1,3galactosyltransferase DNA molecule. The ribozyme will recognized the mRNA of the 1,3galactosyltransferase by complementary base pairing with the mRNA. Subsequently, the ribozyme will cleave and destroy the RNA in a sequence-specific manner, before the enzyme is translated. After dissociation from the cleaved substrate, the ribozyme will repeatedly hybridize with RNA molecules and act as specific endonuclease. In general, ribozymes may specifically be produced for inactivation of a certain mRNA, even if not the entire DNA sequence which codes for the protein is known. Ribozymes are particularly efficient if the ribosomes move slowly along the mRNA. In that case it is easier for the ribozyme to find a ribosome-free site on the mRNA. For this reason, slow ribosome mutants are also suitable as a system for ribozymes (J. Burke, 1997, Nature Biotechnology; 15, 414-415). This DNA molecule is particularly advantageous for the downregulation and inhibition, respectively, of the expression of plant β1,3galactosyltransferases.

One possible way is also to use a varied form of a ribozmye, i.e. a minizyme. Minizymes are efficient particularly for cleaving larger mRNA molecules. A minizyme is a hammer head ribozyme which has a short oligonucleotide linker instead of the stem/loop II. Dimer-minizymes are particularly efficient (Kuwabara et al., 1998, Nature Biotechnology, 16; 961-965).

Consequently, the invention also relates to a biologically functional vector which comprises one of the two last-mentioned DNA molecules (mutation or ribozyme-DNA molecule). What has been said above regarding vectors also applies in this instance. Such a vector can be, for example, inserted into a microorganism and can be used for the production of high concentrations of the above described DNA molecules. Furthermore such a vector is particularly good for the insertion of a specific DNA molecule into a plant organism in order to downregulate or completely inhibit the β1,3galactosyltransferase production in this organism. All vectors described above can also be made with genomic sequences of β1,3-GalT genes, such as SEQ ID NOs. 3 or 4.

Bryophyte cells of the invention or ancestors thereof may be any which have been transformed previously with heterologous genes of interest that code for primary sequences of proteins of interest which are glycosylated with mammalian glycosylation patterns as described herein. Preferably, the glycosylation patterns are of the human type. Alternatively, the bryophyte cell may be transformed severally, that is, simultaneously or over time with nucleotide sequences coding for at least a primary protein sequence of interest, typically at least a pharmaceutical protein of interest for use in humans or mammals such as livestock species including bovine, ovine, equine and porcine species, that require mammalian glycosylation patterns to be placed on them in accordance with the methods of the invention as described herein. Such pharmaceutical glycoproteins for use in mammals, including man include but are not limited to proteins such as VEGF, interferons such as α-interferon, β-interferon, gamma-interferon, blood-clotting factors selected from Factor VII, VIII, IX, X, XI, and XII, fertility hormones including luteinising hormone, follicle stimulating hormone growth factors including epidermal growth factor, platelet-derived growth factor, granulocyte colony stimulating factor and the like, prolactin, oxytocin, thyroid stimulating hormone, adrenocorticotropic hormone, calcitonin, parathyroid hormone, somatostatin, erythropoietin (EPO), enzymes such as β-glucocerebrosidase, haemoglobin, collagen, fusion proteins such as the fusion protein of TNF αreceptor ligand binding domain with Fc portion of IgG and the like. Furthermore, the method of the invention can be used for the production of immunglobulins such as antibodies such as specific monoclonal antibodies or active fragments thereof.

Detailed information on the culturing of mosses which are suitable for use in the invention, such as Leptobryum pyriforme and Sphagnum magellanicum in bioreactors, is known in the prior art (see, for example, E. Wilbert, “Biotechnological studies concerning the mass culture of mosses with particular consideration of the arachidonic acid metabolism”, Ph.D. thesis, University of Mainz (1991); H. Rudolph and S. Rasmussen, Studies on secondary metabolism of Sphagnum cultivated in bioreactors, Crypt. Bot., 3, pp. 67-73 (1992)). Especially preferred for the purposes of the present invention is the use of Physcomitrella patens, since molecular biology techniques are practised on this organism (for a review see R. Reski, Development, genetics and molecular biology of mosses, Bot. Acta, 111, pp. 1-15 (1998)).

Suitable transformation systems have been developed for the biotechnological exploitation of Physcomitrella for the production of heterologous proteins. For example, successful transformations have been carried out by direct DNA transfer into protonema tissue using particle guns. PEG-mediated DNA transfer into moss protoplasts has also been successfully achieved. The PEG-mediated transformation method has been described many times for Physcomitrella patens and leads both to transient and to stable transformants (see, for example, K. Reutter and R. Reski, Production of a heterologous protein in bioreactor cultures of fully differentiated moss plants, Pl. Tissue culture and Biotech., 2, pp. 142-147 (1996)).

In a further embodiment of the present invention there is provided a method of producing at least a bryophyte cell wherein β-1,3-GalT activity is substantially reduced that comprises introducing into the said cell i) a first nucleic acid sequence that is specifically targeted to the endogenous β1,3 encoding nucleotide sequence according to SEQ ID NO: 1 and ii) a second nucleic acid sequence that is specifically targeted to the endogenous β1,3 encoding nucleotide sequence according to SEQ ID NO: 2.

The skilled addressee will appreciate that the order of introduction of said first and second transferase nucleic acid sequences into the bryophyte cell is not important: it can be performed in any order. The first and second nucleic acid sequences can be targeted to specific portions of the endogenous, native β1,3-GalT genes located in the nuclear genome of the bryophyte cell defined by specific restriction enzyme sites thereof, for example, according to the examples as provided herein. By specifically targeting the sequences of the native β1,3-GalT genes with nucleotide sequences that specifically integrate with the target native transferase genes of interest, the expression of the said sequences is substantially impaired if not completely disrupted.

Preferably all glycosylated mammalian proteins mentioned herein-above are of the human type. Other proteins that are contemplated for production in the present invention include proteins for use in veterinary care and may correspond to animal homologues of the human proteins mentioned herein.

An exogenous promoter is one that denotes a promoter that is introduced in front of a nucleic acid sequence of interest and is operably associated therewith. Thus an exogenous promoter is one that has been placed in front of a selected nucleic acid component as herein defined and does not consist of the natural or native promoter usually associated with the nucleic acid component of interest as found in wild type circumstances. Thus a promoter may be native to a bryophyte cell of interest but may not be operably associated with the nucleic acid of interest in front in wild-type bryophyte cells. Typically, an exogenous promoter is one that is transferred to a host bryophyte cell from a source other than the host cell.

Regarding the production of N-glycan structures with improved β1,3-galactosylation the cDNA's encoding the β-1,3-GalT proteins, the glycosylated and the mammalian proteins as described herein contain at least one type of promoter that is operable in a bryophyte cell, for example, an inducible or a constitutive promoter operatively linked to a β-1,3-GalT nucleic acid sequence and/or second nucleic acid sequence for a glycosylated mammalian protein as herein defined and as provided by the present invention. As discussed, this enables control of expression of the gene(s).

The term “inducible” as applied to a promoter is well understood by those skilled in the art. In essence, expression under the control of an inducible promoter is “switched on” or increased in response to an applied stimulus (which may be generated within a cell or provided exogenously). The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus. The preferable situation is where the level of expression increases upon application of the relevant stimulus by an amount effective to alter a phenotypic characteristic. Thus an inducible (or “switchable”) promoter may be used which causes a basic level of expression in the absence of the stimulus which level is too low to bring about a desired phenotype (and may in fact be zero). Upon application of the stimulus, expression is increased (or switched on) to a level, which brings about the desired phenotype.

As alluded to herein, bryophyte expression systems are also known to the man skilled in the art. A bryophyte promoter, in particular a Physcomitrella patens promoter, is any DNA sequence capable of binding a host DNA-dependent RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site (the “TATA Box”) and a transcription initiation site. A bryophyte promoter may also have a second domain called an upstream activator sequence (UAS), which, if present, is usually distal to the structural gene. The UAS permits regulated (inducible) expression. Constitutive expression occurs in the absence of a UAS. Regulated expression may be either positive or negative, thereby either enhancing or reducing transcription.

The skilled addressee will appreciate that bryophyte promoter sequences encoding enzymes in bryophyte metabolic pathways can provide particularly useful promoter sequences.

In addition, synthetic promoters which do not occur in nature may also function as bryophyte promoters. For example, UAS sequences of one byrophyte promoter may be joined with the transcription activation region of another bryophyte promoter, creating a synthetic hybrid promoter. An example of a suitable promoter is the one used in the TOP 10 expression system for Physcomitrella patens by Zeidler et al. (1996) Plant. Mol. Biol. 30, 199-205). Furthermore, a bryophyte promoter can include naturally occurring promoters of non-bryophyte origin that have the ability to bind a bryophyte DNA-dependent RNA polymerase and initiate transcription. Examples of such promoters include those described, inter alia, the rice P-Actin 1 promoter and the Chlamydomonas RbcS promoter (Zeidler et al. (1999) J. Plant Physiol. 154, 641-650), Cohen et al., Proc. Natl. Acad. Sci. USA, 77: 1078, 1980; Henikoff et al., Nature, 283: 835, 1981; Hollenberg et al., Curr. Topics Microbiol. Immunol., 96: 119, 1981; Hollenberg et al., “The Expression of Bacterial Antibiotic Resistance Genes in the Yeast Saccharomyces cerevisiae”, in: Plasmids of Medical, Environmental and Commercial Importance (eds. K. N. Timms and A. Puhler), 1979; Mercerau-Puigalon et al., Gene, 11: 163, 1980; Panthier et al., Curr. Genet., 2: 109, 1980.

The DNA molecules according to the present invention may be expressed intracellularly in bryophytes. A promoter sequence may be directly linked with the DNA molecule, in which case the first amino acid at the N-terminus of the recombinant protein will always be a methionine, which is encoded by the AUG start codon on the mRNA. If desired, methionine at the N-terminus may be cleaved from the protein by in vitro incubation with cyanogen bromide.

Alternatively, foreign proteins can also be secreted from the bryophyte cell into the growth media by creating chimeric DNA molecules that encode a fusion protein comprised of a leader sequence fragment that provides for secretion in or out of bryophyte cells of the foreign protein. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell.

DNA encoding suitable signal sequences can be derived from genes for secreted bryophyte proteins, such as leaders of non-bryophyte origin, such as a VEGF leader, exist that may also provide for secretion in bryophyte cells.

Transcription termination sequences that are recognized by and functional in bryophyte cells are regulatory regions located 3′ to the translation stop codon, and thus together with the promoter flank the coding sequence. These sequences direct the transcription of an mRNA which can be translated into the polypeptide encoded by the DNA. An example of a suitable termination sequence that works in Physcomitrella pa tens is the termination region of Cauliflower mosaic virus.

Typically, the components, comprising a promoter, leader (if desired), coding sequence of interest, and transcription termination sequence, are put together into expression constructs of the invention. Expression constructs are often maintained in a DNA plasmid, which is an extrachromosomal element capable of stable maintenance in a host, such as a bacterium. The DNA plasmid may have two origins of replication, thus allowing it to be maintained, for example, in a bryophyte for expression and in a prokaryotic host for cloning and amplification. Generally speaking it is sufficient if the plasmid has one origin of replication for cloning and amplification in a prokaryotic host cell. In addition, a DNA plasmid may be either a high or low copy number plasmid. A high copy number plasmid will generally have a copy number ranging from about 5 to about 200, and usually about 10 to about 150. A host containing a high copy number plasmid will preferably have at least about 10, and more preferably at least about 20. Either a high or low copy number vector may be selected, depending upon the effect of the vector and the foreign protein on the host (see, e.g., Brake et al., supra).

Alternatively, the expression constructs can be integrated into the bryophyte genome with an integrating vector. Integrating vectors usually contain at least one sequence homologous to a bryophyte chromosome that allows the vector to integrate, and preferably contain two homologous sequences flanking the expression construct. An integrating vector may be directed to a specific locus in moss by selecting the appropriate homologous sequence for inclusion in the vector as described and exemplified herein. One or more expression constructs may integrate. The chromosomal sequences included in the vector can occur either as a single segment in the vector, which results in the integration of the entire vector, or two segments homologous to adjacent segments in the chromosome and flanking the expression construct in the vector, which can result in the stable integration of only the expression construct.

Usually, extrachromosomal and integrating expression constructs may contain selectable markers to allow for the selection of bryophyte cells that have been transformed.

Selectable markers may include biosynthetic genes that can be expressed in the moss host, such as the G418 or hygromycin B resistance genes, which confer resistance in bryophyte cells to G418 and hygromycin B, respectively. In addition, a suitable selectable marker may also provide bryophyte cells with the ability to grow in the presence of toxic compounds, such as metal.

Alternatively, some of the above-described components can be put together into transformation vectors. Transformation vectors are usually comprised of a selectable marker that is either maintained in a DNA plasmid or developed into an integrating vector, as described above.

Alternatively, by achieving high yields of transformation events as observed in Physcomitrella the use of markers for the selection of transformation events can be avoided.

Methods of introducing exogenous DNA into bryophyte cells are well-known in the art, and are described inter alia by Schaefer D. G. “Principles and protocols for the moss Physcomitrella patens”, (May 2001) Institute of Ecology, Laboratory of Plant Cell Genetics, University of Lausanne; Reutter K. and Reski R., Plant Tissue Culture and Biotechnology September 1996, Vol. 2, No. 3; Zeidler M et al., (1996), Plant Molecular Biology 30:199-205.

Those skilled in the art are well able to construct vectors and design protocols for recombinant nucleic acid sequence or gene expression as described above. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

As described above, selectable genetic markers may facilitate the selection of transgenic bryophyte cells and these may consist of chimaeric genes that confer selectable phenotypes as alluded to herein.

When introducing selected glycosyltransferase encoding nucleic acid sequences and polypetide sequences comprising glycosyltransferase activity into a bryophyte cell, certain considerations must be taken into account, well known to those skilled in the art. The nucleic acid(s) to be inserted should be assembled within a construct, which contains effective regulatory elements, which will drive transcription. There must be available a method of transporting the construct into the cell. Once the construct is within the cell membrane, integration into the endogenous chromosomal material either will or will not occur.

The invention further encompasses a host cell transformed with vectors or constructs as set forth above, especially a bryophyte or a microbial cell. Thus, a host cell, such as a bryophyte cell, including nucleotide sequences of the invention as herein indicated is provided. Within the cell, the nucleotide sequence may be incorporated within the chromosome.

Also according to the invention there is provided a bryophyte cell having incorporated into its genome at least a nucleotide sequence, particularly heterologous nucleotide sequences, as provided by the present invention under operative control of regulatory sequences for control of expression as herein described. The coding sequence may be operably linked to one or more regulatory sequences which may be heterologous or foreign to the nucleic acid sequences employed in the invention, such as not naturally associated with the nucleic acid sequence(s) for its(their) expression. The nucleotide sequence according to the invention may be placed under the control of an externally inducible promoter to place expression under the control of the user. A further aspect of the present invention provides a method of making such a bryophyte cell, particularly a Physcomitrella patens cell involving introduction of nucleic acid sequence(s) contemplated for use in the invention or at least a suitable vector including the sequence(s) contemplated for use in the invention into a bryophyte cell and causing or allowing recombination between the vector and the bryophyte cell genome to introduce the said sequences into the genome. The invention extends to bryophyte cells, particularly Physcomitrella patens cells containing a GalT nucleotide and/or a nucleotide sequence coding for a polypeptide sequence destined for the addition of a mammalian glycosylation pattern thereto and suitable for use in the invention as a result of introduction of the nucleotide sequence into an ancestor cell.

The term “heterologous” may be used to indicate that the gene/sequence of nucleotides in question have been introduced into bryophyte cells or an ancestor thereof, using genetic engineering, i.e. by human intervention. A transgenic bryophyte cell, i.e. transgenic for the nucleotide sequence in question, may be provided. The transgene may be on an extra-genomic vector or incorporated, preferably stably, into the genome. A heterologous gene may replace an endogenous equivalent gene, i.e. one that normally performs the same or a similar function, or the inserted sequence may be additional to the endogenous gene or other sequence. An advantage of introduction of a heterologous gene is the ability to place expression of a sequence under the control of a promoter of choice, in order to be able to influence expression according to preference. Nucleotide sequences heterologous, or exogenous or foreign, to a bryophyte cell may be non-naturally occurring in cells of that type, strain or species. Thus, a nucleotide sequence may include a coding sequence of or derived from a particular type of bryophyte cell, such as a Physcomitrella patens cell, placed within the context of a bryophyte cell of a different type or species. A further possibility is for a nucleotide sequence to be placed within a bryophyte cell in which it or a homologue is found naturally, but wherein the nucleotide sequence is linked and/or adjacent to nucleic acid which does not occur naturally within the cell, or cells of that type or species or strain, such as operably linked to one or more regulatory sequences, such as a promoter sequence, for control of expression. A sequence within a bryophyte or other host cell may be identifiably heterologous, exogenous or foreign.

The present invention also encompasses the desired polypeptide expression product of the combination of nucleic acid molecules according to the invention as disclosed herein or obtainable in accordance with the information and suggestions herein. Also provided are methods of making such an expression product by expression from nucleotide sequences encoding therefore under suitable conditions in suitable host cells e.g. E. coli. Those skilled in the art are well able to construct vectors and design protocols and systems for expression and recovery of products of recombinant gene expression.

A polypeptide according to the present invention may be an allele, variant, fragment, derivative, mutant or homologue of the(a) polypeptides as mentioned herein. The allele, variant, fragment, derivative, mutant or homologue may have substantially the same function of the polypeptides alluded to above and as shown herein or may be a functional mutant thereof. In the context of pharmaceutical proteins as described herein for use in humans, the skilled addressee will appreciate that the primary sequence of such proteins and their glycosylation pattern will mimic or preferably be identical to that found in humans.

“Identity” in relation to a nucleic acid sequence or to an amino acid sequence of the invention may be used to refer to identity of the whole sequence or essential parts thereof. As noted already above, high level of amino acid identity may be limited to functionally significant domains or regions, e.g. any of the domains identified herein.

In particular, homologues of the particular bryophyte-derived polypeptide sequences provided herein, are provided by the present invention, as are mutants, variants, fragments and derivatives of such homologues. Thus the present invention also extends to polypeptides which include amino acid sequences with μ1,3-galactosyltransferases function as defined herein and as obtainable using sequence information as provided herein. The β1,3-galactosyltransferase according to the present invention may at the amino acid level have identity with the amino acid sequences of the sequences disclosed herein, especially of PpGalT1, PpGalT2, PpGalT1as or PpGalT2as (FIGS. 2-5), of at least about 50%, or at least 55%, or at least about 60%, or at least about 65%, or at least about 70%, or at least about 75%, or at least about 80% identity, or at least about 85%, or at least about 88% identity, or at least about 90% identity and most preferably at least about 95% or greater identity provided that such proteins have a β1,3-galactosyltransferase activity that fits within the context of the present invention. The % identity mentioned should be preferably given in the region comprising the seven conserved domains as depicted in FIG. 1 (including appropriate “-” as being obvious occurring to the skilled man in the art) when comparing the sequences in question to e.g. either PpGalT1, PpGalT2, PpGalT1as or PpGalT2as.

In certain embodiments, an allele, variant, derivative, mutant derivative, mutant or homologue of the specific sequence may show little overall identity, e.g. at least 20%, or at least 25%, or at least 30%, or at least 35%, or at least 40% or at least 45% (i.e. say about 20%, or about 25%, or about 30%, or about 35%, or about 40%, or about 45% (i.e. being e.g. 20% or above)), with the specific sequence. However, in functionally significant domains or regions, the amino acid identity may be much higher. Putative functionally significant domains or regions can be identified using processes of bioinformatics, including comparison of the sequences of homologues. Preferred β1,3-GalT proteins according to the present invention show more than 80%, especially more than 90% identity in the seven conserved domains according to FIG. 1 (amino acid residues in bold), especially preferred with the conserved amino acids (represented by a “*” (star) in FIG. 1) being completely (or at least to a 95% extent) present. Specifically preferred variants of the β1,3-GalT according to the present invention comprise more than 80%, preferably more than 90%, especially 100%, of the conserved amino acids as depicted in FIG. 1.

Functionally significant domains or regions of different polypeptides may be combined for expression from encoding nucleic acid as a fusion protein. For example, particularly advantageous or desirable properties of different homologues may be combined in a hybrid protein, such that the resultant expression product, with β1,3-galactosyltransferase function, may include fragments of various parent proteins, if appropriate.

Identity may easily be calculated as % value of aligned sequences (including intelligent “-”). Similarity of amino acid sequences may be as defined and determined by the TBLASTN program, of Altschul et al. (1990) J. Mol. Biol. 215: 403-10, which is in standard use in the art. In particular, TBLASTN 2.0 may be used with Matrix BLOSUM62 and GAP penalties: existence: 11, extension: 1. Another standard program that may be used is BestFit, which is part of the Wisconsin Package, Version 8, September 1994, (Genetics Computer Group, 575 Science Drive, Madison, Wis., USA, Wisconsin 53711). BestFit makes an optimal alignment of the best segment of similarity between two sequences. Optimal alignments are found by inserting gaps to maximize the number of matches using the local identity algorithm of Smith and Waterman (Adv. Appl. Math. (1981) 2: 482-489). Other algorithms include GAP, which uses the Needleman and Wunsch algorithm to align two complete sequences that maximizes the number of matches and minimizes the number of gaps. As with any algorithm, generally the default parameters are used, which for GAP are a gap creation penalty=12 and gap extension penalty=4. Alternatively, a gap creation penalty of 3 and gap extension penalty of 0.1 may be used. The algorithm FASTA (which uses the method of Pearson and Lipman (1988) PNAS USA 85: 2444-2448) is a further alternative.

An advantageous method of producing recombinant host cells, in particular plant cells, or plants, respectively, consists in that the DNA molecule according to the present invention, especially comprising an inactivating mutation is inserted into the genome of the host cell, or plant, respectively, in the place of the non-mutant homologous sequence (Schaefer et al., 1997, Plant J.; 11(6):1195-1206). This method thus does not function with a vector, but with a pure DNA molecule. The DNA molecule according to the present invention is inserted into the host e.g. by gene bombardment, microinjection or PEG-mediated direct DNA transfer, to mention just three examples. This DNA molecule binds to the homologous sequence in the genome of the host so that a homologous recombination and thus reception of the deletion, insertion or substitution mutation, respectively, will result in the genome: Expression of the β1,3-galactosyltransferase can e.g. be suppressed or completely blocked, respectively.

A further aspect of the invention relates to plants, plant tissues or plant cells, respectively their β1,3galactosyltransferase activity being less than 50%, in particular less than 20%, particularly preferred 0%, of the β1,3galactosyltransferase activity occurring in natural plants or plant cells, The advantage of these plants or plant cells, respectively, is that the glycoproteins produced by them do not comprise any or hardly comprise any β1,3-bound galactose. If products of these plants, respectively, are taken up by human or vertebrate bodies, there will be no immune reaction to the β1,3 linked galactose epitope.

Preferably, recombinant plants or plant cells, respectively, are provided which have been prepared by one of the methods described above, their β1,3-galactosyltransferase production being suppressed or completely blocked, respectively.

The invention also relates to a PNA molecule comprising a base sequence complementary to the sequence of the DNA molecule according to the invention as well as partial sequences thereof. PNA (peptide nucleic acid) is a DNA-like sequence, the nucleo-bases being bound to a pseudo-peptide backbone. PNA generally hybridizes with complementary DNA-, RNA- or PNA-oligomers by Watson-Crick base pairing and helix formation. The peptide backbone ensures a greater resistance to enzymatic degradation. The PNA molecule thus is an improved antisense agent. Neither nucleases nor proteases are capable of attacking a PNA molecule. The stability of the PNA molecule, if bound to a complementary sequence, comprises a sufficient steric blocking of DNA and RNA polymerases, reverse transcriptase, telomerase and ribosomes. If the PNA molecule comprises the above-mentioned sequence, it will bind to the DNA or to a site of the DNA, respectively, which codes for β1,3galactosyltransferase and in this way is capable of inhibiting transcription of this enzyme. As it is neither transcribed nor translated, the PNA molecule will be prepared synthetically, e.g. by aid of the t-Boc technique. Advantageously, a PNA molecule is provided which comprises a base sequence which corresponds to the sequence of the inventive DNA molecule as well as partial sequences thereof. This PNA molecule will complex the mRNA or a site of the mRNA of β1,3-galactosyltransferase so that the translation of the enzyme will be inhibited. Similar arguments as set forth for the antisense RNA apply in this case. Thus, e.g., a particularly efficient complexing region is the translation start region or also the 5′-non-translated regions of mRNA.

A further aspect of the present invention relates to a method of preparing plants, tissues, or cells, respectively, in particular plant cells which comprise a blocked expression of the β1,3galactosyltransferase on transcription or translation level, respectively, which is characterized in that inventive PNA molecules are inserted in the cells. To insert the PNA molecule or the PNA molecules, respectively, in the cell, again conventional methods, such as, e.g., electroporation or microinjection, are used. Particularly efficient is insertion if the PNA oligomers are bound to cell penetration peptides, e.g. transportan or pAntp (Pooga et al., 1998, Nature Biotechnology, 16; 857-861).

The invention provides a method of preparing recombinant glycoproteins which is characterized in that the inventive, recombinant plants or plant cells, respectively, whose β1,3-galactosyltransferase production is suppressed or completely blocked, respectively, or plants, or tissues, or cells, respectively, in which the PNA molecules have been inserted according to the method of the invention, are transfected with the gene that expresses the glycoprotein so that the recombinant glycoproteins are expressed. In doing so, as has already been described above, vectors comprising genes for the desired proteins are transfected into the host or host cells, respectively, as has also already been described above. The transfected plant cells will express the desired proteins, and they have no or hardly any β1,3-bound galactose. Thus, they do not trigger the immune reactions already mentioned above in the human or vertebrate body. Any proteins may be produced in these systems.

Advantageously, a method of preparing recombinant human glycoproteins is provided which is characterized in that the recombinant plants or plant cells, respectively, whose β1,3-galactosyltransferase production is suppressed or completely blocked, or plants, or tissues, or cells, respectively, in which PNA molecules have been inserted according to the method of the invention, are transfected with the gene that expresses the glycoprotein so that the recombinant glycoproteins are expressed. By this method it becomes possible to produce human proteins in plants (plant cells) which, if taken up by the human body, do not trigger any immune reaction directed against β1,3-bound galacatase residues. There, it is possible to utilize plant types for producing the recombinant glycoproteins which serve as food stuffs, e.g. banana, potato and/or tomato. The tissues of this plant comprise the recombinant glycoprotein so that, e.g. by extraction of the recombinant glycoprotein from the tissue and subsequent administration, or directly by eating the plant tissue, respectively, the recombinant glycoprotein is taken up in the human body. Preferably, a method of preparing recombinant human glycoproteins for medical use is provided, wherein the inventive, recombinant plants or plant cells, respectively, whose β1,3-galactosyltransferase production is suppressed or completely blocked, respectively, or plants, or tissues, or cells, respectively, into which the PNA molecules have been inserted according to the method of the invention, are transfected with the gene that expresses the glycoprotein so that the recombinant glycoproteins are expressed. In doing so, any protein can be used which is of medical interest.

Moreover, the present invention relates to recombinant glycoproteins according to a method described above, wherein they have been prepared in plant systems and wherein their peptide sequence comprises less than 50%, in particular less than 20%, particularly preferred 0%, of the β1,3-bound galactose residues occurring in proteins expressed in non-galactosyltransferase-reduced plant systems. Naturally, glycoproteins which do not comprise β1,3-bound galactose residues are to be preferred. The amount of β1,3-bound galactose will depend on the degree of the above-described suppression of the β1,3-galactosyltransferase. Preferably, the invention relates to recombinant human glycoproteins which have been produced in plant systems according to a method described above and whose peptide sequence comprises less than 50%, in particular less than 20%, particularly preferred 0%, of the β1,3-bound galactose residues occurring in the proteins expressed in non-galactosyltransferase-reduced plant or systems.

A particularly preferred embodiment relates to recombinant human glycoproteins for medical use which have been prepared in plant systems according to a method described above and whose peptide sequence comprises less than 50%, in particular less than 20%, particularly preferred 0%, of the β1,3-bound galactose residues occurring in the proteins expressed in non-galactosyltransferase-reduced plant systems.

A further aspect comprises a pharmaceutical composition comprising the glycoproteins according to the invention. In addition to the glycoproteins of the invention, the pharmaceutical composition comprises further additions common for such compositions. These are, e.g., suitable diluting agents of various buffer contents (e.g. Tris-HCl, acetate, phosphate, pH and ionic strength, additives, such as tensides and solubilizers (e.g. Tween 80, Polysorbate 80), preservatives (e.g. Thimerosal, benzyl alcohol), adjuvants, antioxidants (e.g. ascorbic acid, sodium metabisulfite), emulsifiers, fillers (e.g. lactose, mannitol), covalent bonds of polymers, such as polyethylene glycol, to the protein, incorporation of the material in particulate compositions of polymeric compounds, such as polylactic acid, poly-glycolic acid, etc. or in liposomes, auxiliary agents and/or carrier substances which are suitable in the respective treatment. Such compositions will influence the physical condition, stability, rate of in vivo liberation and rate of in vivo excretion of the glycoproteins of the invention.

The invention also provides a method of selecting DNA molecules which code for a β1,3-galactosyltransferase, in a sample, wherein the labelled DNA molecules of the invention are admixed to the sample, which bind to the DNA molecules that code for a β1,3-galactosyltransferase. The hybridized DNA molecules can be detected, quantitated and selected. For the sample to contain single strand DNA with which the labelled DNA molecules can hybridize, the sample is denatured, e.g. by heating.

One possible way is to separate the DNA to be assayed, possibly after the addition of endonucleases, by gel electrophoresis on an agarose gel. After having been transferred to a membrane of nitrocellulose, the labelled DNA molecules according to the invention are admixed which hybridize to the corresponding homologous DNA molecule (“Southern blotting”).

Another possible way consists in finding homologous genes from other species by PCR-dependent methods using specific and/or degenerated primers, derived from the sequence of the DNA molecule according to the invention.

Preferably, the sample for the above-identified inventive method comprises genomic DNA of a plant organism. By this method, a large number of plants is assayed in a very rapid and efficient manner for the presence of the β1,3-galactosyltransferase gene. In this manner, it is respectively possible to select plants which do not comprise this gene, or to suppress or completely block, respectively, the expression of the β1,3-galactosyltransferase in such plants which comprise this gene, by an above-described method of the invention, so that subsequently they may be used for the transfection and production of (human) glycoproteins.

The invention also relates to DNA molecules which code for a β1,3-galactosyltransferase which have been selected according to the two last-mentioned methods and subsequently have been isolated from the sample. These molecules can be used for further assays. They can be sequenced and in turn can be used as DNA probes for finding β1,3-galactosyltransferases. These—labelled—DNA molecules will function for organisms, which are related to the organisms from which they have been isolated, more efficiently as probes than the DNA molecules of the invention.

The invention also relates to a method of preparing “plantified” carbohydrate units of human and other vertebrate glycoproteins, wherein fucose units as well as β1,3galactosyltransferase encoded by an above-described DNA molecule are admixed to a sample that comprises a carbohydrate unit or a glycoprotein, respectively, so that galactose in β1,3-position will be bound by the β1,3galactosyltransferase to the carbohydrate unit or to the glycoprotein, respectively. By the method according to the invention for cloning β1,3galactosyltransferase it is possible to produce large amounts of purified enzyme. To obtain a fully active transferase, suitable reaction conditions are provided.

The invention will be explained in more detail by way of the following examples and drawing figures to which, of course, it shall not be restricted.

FIG. 1 shows an amino acid alignment of β1,3-GalT. The seven conserved domains of β1,3-galactosyltransferases are indicated in bold letters. Conserved amino acid residues are indicated by stars. Similarities according to the reference sequence from humans (CAA75344, β1,3-galactosyltransferase from humans) are predicted as follows BAD17812 (putative β1,3-galactosyltransferase from Oryza sativa)=17%; NP 174003 (putative β1,3-galactosyltransferase from Arabidopsis thaliana)=16%; PpGalT1 (β1,3-galactosyltransferase 1 from Physcomitrella patens)=15%; PpGalT2 (β1,3-galactosyltransferase 2 from Physcomitrella patens)=16%;

FIG. 2 shows the protein sequence predicted from the coding DNA sequence of the β1,3-galactosyltransferase 1 gene from Physcomitrella patens. The transmembrane domain is indicated in bold letters; and

FIG. 3 shows the protein sequence predicted from the coding DNA sequence of the β1,3-galactosyltransferase 2 gene from Physcomitrella patens. The transmembrane domain is indicated in bold letters.

FIG. 4 shows the protein sequence of an alternative splice variant of the β1,3-galactosyltransferase 1 gene from physcomitrella patens. The additional 55 amino acid splice insert is indicated in bold letters.

FIG. 5 shows the protein sequence of an alternative splice variant of the β1,3-galactosyltransferase 2 gene form P. patens. The additional 50 amino acid splice insert is indicated in bold letters.

### EXAMPLES

### Methods and Materials

### Plant Material

A glyco-engineered double knockout strain of Physcomitrella patens lacking fucose and xylose residues in the core structure of N-glycans was used (Koprivova et al. 2004 Plant Biotechnol. J. 2, 517-523).

### Standard Culture Conditions

Plants were grown axenicallly under sterile conditions in plain inorganic liquid modified Knop medium (1000 mg/l Ca(NO₃)₂×4H₂O 250 mg/l KCl, 250 mg/l KH₂PO4, 250 mg/l MgSO₄×7H₂O and 12.5 mg/l FeSO₄×7H₂O; pH 5.8 (Reski and Abel (1985) Planta 165, 354-358). Plants were grown in 500 ml Erlenmeyer flasks containing 200 ml of culture medium and flasks were shaken on a Certomat R shaker (B. Braun Biotech International, Germany) set at 120 rpm. Conditions in the growth chamber were 25+/−3° C. and a light-dark regime of 16:8 h. The flasks were illuminated from above by two fluorescent tubes (Osram L 58 W/25) providing 35 micromols⁻¹m⁻². The cultures were subcultured once a week by disintegration using an Ultra-Turrax homogenizer (IKA, Staufen, Germany) and inoculation of two new 500 ml Erlenmeyer flasks containing 100 ml fresh Knop medium.

### Protoplast Isolation

After filtration the moss protonemata were preincubated in 0.5 M mannitol. After 30 min, 4% Driselase (Sigma, Deisenhofen, Germany) was added to the suspension. Driselase was dissolved in 0.5 M mannitol (pH 5.6-5.8), centrifuged at 3600 rpm for 10 min and sterilised by passage through a 0.22 microm filter (Millex GP, Millipore Corporation, USA). The suspension, containing 1% Driselase (final concentration), was incubated in the dark at RT and agitated gently (best yields of protoplasts were achieved after 2 hours of incubation) (Schaefer, “Principles and protocols for the moss Physcomitrella patens”, (May 2001) Institute of Ecology, Laboratory of Plant Cell Genetics, University of Lausanne. The suspension was passed through sieves (Wilson, CLF, Germany) with pore sizes of 100 microm and 50 microm. The suspension was centrifuged in sterile centrifuge tubes and protoplasts were sedimented at RT for 10 min at 55 g (acceleration of 3; slow down at 3; Multifuge 3 S-R, Kendro, Germany) (Schaefer, supra). Protoplasts were gently resuspended in 3M medium (15 mM MgCl₂×2H₂O; 0.1% MES; 0.48 M mannitol; pH 5.6; 540 mOsm; sterile filtered, Schaefer et al. (1991) Mol Gen Genet 226, 418-424). The suspension was centrifuged again at RT for 10 min at 55 g (acceleration of 3; slow down at 3; Multifuge 3 S-R, Kendro, Germany). Protoplasts were gently resuspended in 3M medium (15 mM MgCl₂×2H₂O; 0.1% MES; 0.48 M mannitol; pH 5.6; 540 mOsm; sterile filtered, Schaefer et al. (1991) Mol Gen Genet 226, 418-424). For counting protoplasts a small volume of the suspension was transferred to a Fuchs-Rosenthal-chamber.

### Transformation Protocol

For transformation protoplasts were incubated on ice in the dark for 30 minutes. Subsequently, protoplasts were sedimented by centrifugation at RT for 10 min at 55 g (acceleration of 3; slow down at 3; Multifuge 3 S-R, Kendro). Protoplasts were resuspended in 3M medium (15 mM MgCl₂×2H₂O; 0.1% MES; 0.48 M mannitol; pH 5.6; 540 mOsm; sterile filtered, Schaefer et al. (1991) Mol Gen Genet 226, 418-424) at a concentration of 1.2×10⁶ protoplasts/ml (Reutter and Reski (1996) Production of a heterologous protein in bioreactor cultures of fully differentiated moss plants, Pl. Tissue culture and Biotech., 2, pp. 142-147). 25 microlitre of this protoplast suspension were dispensed into a new sterile centrifuge tube, 5 microlitre DNA solution (column purified DNA in H₂O (Qiagen, Hilden, Germany); 10-100 microlitre; optimal DNA amount of 6 microgram) was added and finally 25 microlitre PEG-solution (40% PEG 4000; 0.4 M mannitol; 0.1 M Ca(NO₃)₂; pH 6 after autoclaving) was added. The suspension was immediately but gently mixed and then incubated for 6 min at RT with occasional gentle mixing. The suspension was diluted progressively by adding 1, 2, 3 and 4 ml of 3M medium. The suspension was centrifuged at 20° C. for 10 minutes at 55 g (acceleration of 3; slow down at 3; Multifuge 3 S-R, Kendro). The pellet was resuspended in 3 ml regeneration medium (modified Knop medium; 5% glucose; 3% mannitol; 540 mOsm; pH 5.6-5.8). Regeneration was performed as described by Strepp et al. (1998) Proc Natl Acad Sci USA 95, 4368-4373). Transgenic clones were identified by molecular screening.

### MALDI-Tof MS of Moss Glycans

Plant material was cultivated in liquid culture, isolated by filtration, frozen in liquid nitrogen and stored at −80° C. The material was shipped under dry ice. The MALDI-TOF MS analyses were done in the laboratory of Prof. Dr. F. Altmann, Glycobiology Division, Institut für Chemie, Universität für Bodenkultur, Vienna, Austria.

0.2 to 0.5 g fresh weight of transgenic Physcomitrella patens material was digested with pepsin. N-glycans were obtained from the digest as described by Wilson et al. (2001). Essentially, the glycans were released by treatment with peptide:N-glycosidase A and analysed by MALDI-TOF mass spectrometry on a DYNAMO (Thermo BioAnalysis, Santa Fe, N. Mex.).

1. Identification of β1,3-galactosyltransferase Encoding Genes

Although biological functionality β1,3-galactosyltransferases (β-1,3galT) from humans in respect to the elongation of N-glycan structures was not described the sequence of the β-1,3galT 2 (Acc.No: CAA75344) of humans was chosen as starting sequence. Based on the seven conserved domains described by Hennet (2002 Cell. Mol. Life Sci. 59, 1081-1095) and in combination with the conserved amino acids described by Amado et al. (1998 J. Biol. Chem. 273, 12770-12778) a database screening was performed. Due to this strategy one sequence from Arabidopsis thaliana (Acc.No: NP174003) and one sequence from Oryza sativa (Acc.No: BAD17812) described as putative β1,3-galactosyltransferases were identified. Although for both species numerous protein sequences of putative β1,3-galactosyltransferases were listed in the public databases only these two showed similarities on the one hand for the seven conserved domains and on the other hand for several of the highly conserved additional amino acids. However, if compared to CAA75344 the overall identity was very low for both, in case of NP174003 it was 16%, in case of BAD17812 it was 17% (FIG. 1).

All three protein sequences were used for the screening of a non public “expressed sequence tag” (EST) database of Physcomitrella patens. An expressed sequence tag encoding a peptide sequence which comprised some similarities with the seven conserved domains of the β1,3-galactosyltransferases was identified. This EST was used to design primers for cloning purposes and for further screening in regard of a beta 1,3-galactosyltransferase gene family of a database comprising genomic sequences of Physcomitrella patens.

The resulting sequences comprised two putative β1,3-galactosyltransferase genes including intron and exon sequences and the gene structures (β-1,3galT 1 corresponds to SEQ ID NO: 1 and SEQ ID NO:3 and β-1,3galT 2 corresponds to SEQ ID NO: 2 and SEQ ID NO: 4). The protein sequences predicted from the open reading frames (β1,3-GalT 1 (FIG. 2) and β1,3-GalT 2 (FIG. 3) comprised transmembrane domains, the seven conserved domains and numerous of the conserved amino acids (FIG. 1).

1.1 Cloning of the Coding Sequence of β1,3-Galactosyltransferase 1 Gene from Physcomitrella patens

Amplification of the nucleotide sequence encoding β1,3-galactosyltransferase from Physcomitrella patens

(SEQ ID NO: 1:
5′AGTTGTCGATTTGTTGTTTTTGATATGTAAGGCGGT-
TGCCTTCGCGCCGTGCTTGATTGTAATTGTAATTCAATCTGGAGTGTGAGATATATATATATA-
TATATATATAGCGAGAGGGAGAGAGAAAGAGAGAGAGAGGGAGAGAGAAAGAGAGAGAGAGG-
GAGAGAGAGAGATGGCTTGTGTATGAGGGCCATGCGAGGAGGAGGCTGTGTTTGTTGCCCGAA-
GAGATGGGATGGTTTATGTGTAGTGCAGGGGTTGGATGTGAAGCACCTGTTTGAAGGAGTCT-
GCGAGAGTTTGAAATTCGGATTCAGAGTGCGGCGATCGATGGTGCAACGTTGTTAGCAGTGAT-
TGTTTTCGCCAACAGAACTGACATCATTTGGATTTTTTTTACGCGTGGATGTGC-
CCTCTTTTTAAAAAATTTCCGCGTGGAANAGAGACGGGGGTTTGTAATGGAGGCAGGCTGTG-
GTCATCACCCCTAGTATAGCCTGTCAAGAGAGTTCAAATTCGGTAATATGAAGAGGGGGTC-
GAGACTACCGGATATGGCGTGTACAGGGCGGCAAAGAAATGATCTTATCCTAGTTGCAAT-
TGTTTGCTTGTTTTTTATGGTGATATTCATCCCACCATATCTCCAAATGAACTCACTTCCGGA-
CATTGATTCTC
CTGATTCGGACAAGAAATCATCAAGCTACTCGAAAAAAACCACTCTAGAAGCCAATAGTAAG-
GAGGAACGCCGTAGTCCGGGGAATACCACAGGCGACATTGTTTCTCTGGATGATGTGATAG-
ATCGTGCCTGGTCTGCTGGTGCCAAAGCGTGGGAAGAACTGGAAACTGCGTTAAGAAATG-
GAGAAGGTGTCTCAAAGAATGTCAGTAATGCCACTGCAAATGCTGATCCGTCTCCAGCAT-
CACTCTCTGCAGCAGGGAAAAAGTTAGACGAATTGGGTAAAGTCTTCCCCTTGCCCTGTG-
GTCTAATGTTTGGGTCAGCCATTACTCTGATTGGAAAGCCTCGAGAGGCTCACATG-
GAGTACAAACCGCCAATCGCCAGAGTTGGGGAAGGCGTCTCTCCATATGTCATG-
GTTTCCCAGTTCTTAGTAGAGTTACAAGGCTTAAAGGTGGTGAAAGGTGAAG-
ATCCTCCTCGAATTCTACACTTGAATCCTCGACTTCGTGGTGATTGGAGCTGGAAACCCAT-
CATTGAGCACAACACTTGTTATCGGAACCAGTGGGGTCCTGCCCACCGATGCGAGGGTTG-
GCAAGTGCCTGAATACGAAGAAACTGTTGACGGTCTTCCCAAGTGCGAGAAGTGGCTTCGAG-
ATGATGGCAAGAAACCTGCTTCAACGCAAAAATCTTGGTGGCTTGGAAGATTAGTTG-
GTCGTTCTGACAAGGAGACGCTTGAATGGGAGTACCCATTATCTGAGGGTCGG-
GAGTTCGTTCTCACCATTCGAGCAGGTGTTGAAGGGTTTCATGTGACTATCGATGGTCGTCA-
CATCAGCTCGTTTCCTTATCGTGTGGGTTACGCTGTGGAAGAAACAACGGGGATA-
TTAGTAGCAGGAGACGTTGATGTGATGTCTATCACAGTGACATCCCTACCCTTAACACATCC-
TAGCTACTACCCTGAGTTAGTTTTGGAATCGGGGGACATTTGGAAGGCACCACCTGTCCCAGC-
TACCAAGATAGATTTATTTATTGGGATCATGTCCAGCAGTAACCATTTTGCAGAACGGATG-
GCAGTAAGGAAGACGTGGTTTCAATCTAAAGCTATTCAATCTTCGCAGGCCGTG-
GCTCGCTTCTTTGTAGCTCTGCATGCAAACAAGGATATCAATATGCAGTTGAAGAAGGAG-
GCAGACTATTATGGCGATATTATAATCCTGCCTTTCATCGACAGATATGATATAGTGGTTCT-
CAAGACCGTTGAAATTTGCAAGTTTGGGGTCCAGAATGTCACAGCTAAGTATATTATGAAGT-
GTGACGATGACACTTTTGTGAGGATTGATAGCGTTCTCGAAGAGATTCGAACTACTTCAATA-
TCACAAGGCCTTTACATGGGTAGCATGAATGAGTTTCACAGGCCTCTTCGTTCTGGAAAGTGG-
GCCGTGACTGCCGAGGAATGGCCTGAGCGAATTTACCCAATATATGCTAATGGACCAGGATA-
TATCCTGTCAGAGGATATTGTGCATTTCATTGTGGAGATGAATGAGAGAGGCAGTTTGCAGT-
TATTTAAGATGGAGGACGTCAGTGTTGGAATATGGGTACGCGAATATGCGAAGCAAGT-
GAAGCACGTTCAATACGAACATAGCATACGGTTTGCTCAAGCCGGTTGTATACCGAAATACT-
TGACAGCTCATTACCAATCGCCGCGTCAAATGCTGTGTCTGTGGGACAAGGTACTTGCTCAT-
GACGATGGGAAATGCTGCAACTTGTGAGGAAAATACATACAATGAATGTCTTCAACG-
GTCTTTACCAGACAGAATTACTTTGGGTCGGGAACCAGATATAGCAGACAGCTCA-
CATTCAATTCAGCCGTGTTGATCCAGAGGGGTAATTGATAGTTTCCTTGTCCCCTACCCTCTC-
TAGAGGTGGAGATCTTACAACTTAATCAAATGATCCTCTGCAATGTCACTTGTCACAATACT-
TAGTATAGCTCAAAATTGGCCACGGATATTCAGGAATGTTCATCTTGTAAGGTCGCAGCTTGT-
GAGTAAATGGTTGGGTGGTGTCGATGGCATGGTTGCTTATCAATCCCTCTTAGCATCAGTG-
ATCGTCAGAATCAGTGTTTTCGACACTCCCCGGTGGAGTATTTTTTCGATTCTCT-
TGATTCCACTCAAGTGGTACTAGCTTATATTTAGTGAGGCCTGGAACCCAAGTAGT-
TAGTTCAGTACGTCTGCCTTTTGCCGAAATGAGTAGAGTAATTTGTGGCAGTAGTTGGTGAA-
GAGACATGGTTAGGATTTAGTGTTCAAAATCTG 3′;

start and stop codon are indicated in bold letters) was performed by PCR with cDNA and the primers MOB1251, (SEQ ID NO: 5: 5′-CTGAATATCCGTGGCCAA-3′) and primer MOB 1410 (SEQ ID NO: 6: 5′-TTCGAGCTCATGAAGAGGGGGTCGAGACT-3′). The amplification product was digested with Sac I and Msc I and cloned into the Sac I/Sma I digested vector pRT101 (Toepfer et al. 1987 NAR 15, 5890). The cloned sequence was verified by sequencing.
1.2 Cloning of the Coding Sequence of β1,3-Galactosyltransferase 2 Gene from Physcomitrella patens

Amplification of the nucleotide sequence encoding β1,3-galactosyltransferase from Physcomitrella patens

(SEQ ID NO: 2:
5′-
ATGAAGAGGGGTGTGAGACCACCGGGTGTGGGATGTACAGGGCGGCAAAGAAACAATCTAAT-
CATAGTGGCAATCATATGTTTGGTTTTTATAGCGATATTCATCCCACCGTTTCTTGAAAT-
GAATTCACTTCCCGATATTGATTCCCCTGTTTTGGAGAAGAAAGTAT-
CAAGCTATTTGAAAAAAGTCACTCTGGAAACTTACAGTAAAGAGGAACGCCGTAGTCCAGG-
GAACACAACAGGTGACATTGTTTCGCTGGAAGATGTGATAGATCGCGCCTGGTCTGCCGGCGC-
CAAAGCTTGGGAAGAGCTGGAAATTGCATTCAGACAGGGAGAACATTTTTCGAAGAAG-
GACAATAATGCCAATGCAACTGCAGATCCATGCCCAGCATCACTCTTTACAACAGGAAAG-
GAATTGGACAATTTAGGAAGGGTCTTCCCACTGCCTTGTGGTCTAATGTTTGGATCAGC-
CATAACTCTCATTGGAAAGCCACGGGAAGCTCACATGGAGTACAAACCGCCAATCGCCAGAGT-
TGGGGAAGGTGTCTCTCCATACGTCATGGTGTCCCAGTTCATAATGGAGTTACAGGGCT-
TGAAGGTG
GTAAAAGGTGAAGATCCTCCTAGAATCCTCCACATAAACCCTCGACTCCGTGGTGACTG-
GAGCTGGAAACCCATCATTGAGCATAATACATGCTATCGAAACCAGTGGGGCCCAGCTCATCG-
GTGTGAAGGTTGGCAAGTACCTGAATACGAAGAAACCGTGGACGGTCTTCCCAAGTGC-
GAGAAGTGGCTTCGAGGCGATGACAAAAAACCTGCTTCGACCCAAAAATCCTGGTGGCTTGG-
GCGATTAGTTGGTCATTCCGACAAGGAGACGCTTGAATGGGAGTATCCATTGTCCGAAG-
GTCGGGAGTTTGTTCTCACCATTCGAGCAGGTGTAGAAGGATTTCACTTAACTATTGATG-
GTCGGCACATCAGTTCGTTCCCTTATCGTGCGGGTTATGCTATGGAAGAAGCAACAGGAATA-
TCAGTGGCAGGAGACGTCGATGTTCTTTCGATGACAGTAACATCATTACCTTTAACA-
CATCCCAGCTACTACCCTGAGTTGGTTTTGGATTCGGGTGATATCTGGAAGGCAC-
CACCTTTACCAACAGGCAAGATAGAGTTATTTGTTGGAATCATGTCAAGCAGCAAT-
CACTTTGCAGAACGTATGGCAGTAAGAAAGACGTGGTTTCAGTCTCTGGT-
TATCCAATCCTCCCAAGCGGTGGCTCGCTTCTTTGTAGCTCTGCATGCAAACAAGGATA-
TCAATCTGCAGCTGAAGAAAGAGGCTGACTATTACGGCGATA-
TGATAATTTTACCTTTCATCGACAGATATGATATAGTGGTTCTTAAGACCGT-
TGAAATTTTCAAGTTTGGGGTCCACAATGTTACAGTTAGCCACGTCATGAAATGTGACGAT-
GACACATTTGTAAGGATTGACAGCGTTCTTGAAGAGATTCGAACGACGTCAGTAGGACAGG-
GCCTTTACATGGGCAGCATGAATGAGTTTCATAGACCCCTTCGTTCTGGGAAGTGGGCCGT-
GACAGTTGAGGAGTGGCCTGAGCGCATTTACCCAACATACGCAAATGGTCCAGGATA-
CATCCTTTCGGAAGATATTGTGCATTTTATAGTGGAGGAGAGCAAAAGAAATAATTTGAGGT-
TATTTAAGATGGAGGACGTCAGCGTAGGTATATGGGTACGCGAGTATGCAAAGAT-
GAAGTACGTGCAATACGAGCATAGCGTACGGTTTGCTCAAGCCGGTTGTATACCTAACTACCT-
GACAGCGCACTATCAATCGCCGCGTCAAATGCTGTGTCTGTGGGACAAGGTGCTTGCTAC-
CAATGACGGCAAGTGCTGCACCTTGTGA -3′;

start and stop codon are indicated in bold letters) was performed by PCR with cDNA and the primers Ppβ1-3 GalT2 for (SEQ ID NO: 7: 5′-TACGAGCTCATGAAGAGGGGTGTGAGACC-3′) and primer Ppβ1-3GalT2 rev (SEQ ID NO: δ: 5′-GTAGAGCTCTCACAAGGTGCAGCACTTG-3′). The amplification product was digested with Sac I and cloned into the Sac I digested vector pRT101 (Toepfer et al. 1987 NAR 15, 5890). The cloned sequence was verified by sequencing.
2.1 Creating the Knockout Construct of the β1,3-Galactosyltransferase 1 Gene from Physcomitrella patens

The knockout construct for targeted gene disruption of the β1,3-galactosyltransferase 1 gene of Physcomitrella patens was generated by PCR performed with genomic DNA from Physcomitrella patens. In one PCR primer MOB1336 (SEQ ID NO: 9: 5′-TACGGATCCAACTTCGAGTTCGTGTCTGTA-3′) and primer MOB1333 (SEQ ID NO: 10: 5′-ACACTAAGCTTCTAATCAATGTCCGGAAGTGAG-3′) were used to amplify the 5′ part of the knockout construct. In a second PCR primer MOB1334 (SEQ ID NO: 11: 5′-TTAGAAGCTTAGTGTACGCTGAGTGTCTACATTG-3′) and primer MOB1335 (SEQ ID NO: 12: 5′-CATTGTCGACCCTACACAGCTCTTAACGTCTAC-3′) were used to amplify the 3′ part of the knockout construct. Both amplified constructs were digested with Hin dIII (restriction sites are indicated in the primer sequences MOB1333 and MOB1334 in bold letters) and were ligated in a subsequent ligation reaction using T4 DNA ligase. The resulting ligated and purified DNA sequence was used as template for a further PCR with primer MOB1336 and MOB1335. The resulting amplification product β1-3GalT1ko

(SEQ ID NO: 13:
5′- CAACTTC-
GAGTTCGTGTCTGTATGAAGAAGTCCACGGGTTCAATGTGTTAAGACTTAGGC-
ATTTCCTTCAGCTTTGCCTAGTGGAGATATGCGTATTTTTTGATTGTGAGGATTCCGGTTCT-
TAGACCATGATTGGTTTATTACAGTGGTCATTCAAATCCTATTTGATTTGAGAAT-
GTATTTACTTCGTTGTGTTGGGAGATGATTGTTCCCTCGAATTCTATGCGGTAGCTAC-
CGCTTCTTTCGTAATGAAGACCTTTGAAGTTCACATAGACTTCAAGAAGAATGCTATTTGT-
GTTTTTGTGATTGTGTGTTCAAGTTTGGTGCAGTATTGTTAAAATTTGGGTGAT-
GACTAAGTACACTTTATGCGGCCCAAGTAGTCAAGTTGAGCATTTGTAAATGCTGAAATGAGT-
TAGGCTGACGGTAAATGTCTGTGGATGTAGCCTAGTGATGTATTTGATCTCG-
GCATAATCTTCAGTGATCAATACAAATAATTCAAGAAAGAGGGGTCAATGTGTTCCTGC-
GAGTACCTTCGCATGTTCAACGTGAACTGAATTATGTTAATTAAGCTGAGCAA-
CATAGACCTTCTTGCTGTTGACAGAGTTCAAATTCGGTAATATGAAGAGGGGGTCGAGACTAC-
CGGATATGGCGTGTACAGGGCGGCAAAGAAATGATCTTATCCTAGTTGCAATTGTTTGCT-
TGTTTTTTATGGTGATATTCATCCCACCATATCTCCAAATGAACTCACTTCCGGACAT-
TGATTAGAAGCTTAGTGTACGCTGAGTGTCTACATTGTGTATTGAATGTTCCTTAGAAT-
TGTTTGTTTGTTTATGTTTTTATTTTTATATTTCTGCCGGCTATTGAGGAAGAATA-
CATTCAAATTGTTCAGGATTCGGACAAGAAATCATCAAGCTACTCGAAAAAAACCACTCTA-
GAAGCCAATAGTAAGGAGGAACGCCGTAGTCCGGGGAATACCACAGGCGACATTGTTTCTCTG-
GATGATGTGATAGATCGTGCCTGGTCTGCTGGTGCCAAAGCGTGGGAAGAACTGGAAACT-
GCGTTAAGAAATGGAGAAGGTGTCTCAAAGAATGTCAGTAATGCCACTGCAAATGCTG-
ATCCGTGTCCAGCATCACTCTCTGCAGCAGGGAAAAAGTTAGACGAATTGG-
GTAAAGTCTTCCCCTTGCCCTGTGGTCTAATGTTTGGGTCAGCCATTACTCTGATTG-
GAAAGCCTCGAGAGGCTCACATGGAGTACAAACCGCCAATCGCCAGAGTTGGGGAAG-
GCGTCTCTCCATATGTCATGGTTTCCCAGTTCTTAGTAGAGTTACAAGGCTTAAAGGTGGT-
GAAAGGTGAAGATCCTCCTCGAATTCTACACTTGAATCCTCGACTTCGTGGTGATTGGAGCTG-
GAAACCCATCATTGAGCACAACACTTGTTATCGGAACCAGTGGGGTCCTGCCCACCGATGC-
GAGGGTTGGCAAGTGCCTGAATACGAAGAAACTGGTGAGTGCTGATTCCACCGCAC-
CAGTTTGTGTTTTTTATGCTGACACTATGCTTCTCAGGTTTGTAGACGTTAAGAGCTGTGTAGG-
3′;

Hin dIII restriction site is indicated in bold letters) comprised a deletion of 270 bp in regard to the genomic sequence of the β1,3-galactosyltransferase gene 1 of Physcomitrella patens which in addition initiate a stop codon in the early 5′ part of the corresponding cDNA. Thus, resulting in a dysfunctional β1,3-galactosyltransferase gene when integrated via homologous recombination into the genome of Physcomitrella patens. This knockout construct was used for transformation of Physcomitrella patens alone or in combination with knockout construct β1-3GalT2ko (see 2.2).

Screening of putative transformed plants was performed by PCR using appropriate primer combinations.

2.2 Creating the Knockout Construct of the β1,3-Galactosyltransferase 2 Gene from Physcomitrella patens

The knockout construct for targeted gene disruption of the β1,3-galactosyltransferase 2 gene of Physcomitrella patens was generated by PCR performed with genomic DNA from Physcomitrella patens. In one PCR primer MOB1339 (SEQ ID NO: 14: 5′-TGGCACGATACAGTGGCATGA-3′) and primer MOB1337 (SEQ ID NO: 15: 5′-TGGAATTCATTCAAGAAACGGTGGGATGA-3′) were used to amplify the 5′ part of the knockout construct. In a second PCR primer MOB1338 (SEQ ID NO: 16: 5′-TGAATTCCATAACGAAGACACCGTCTA-3′) and primer MOB1313 (SEQ ID NO: 17: 5′-CAAGCAGCGGAGACCTTGCAATGC-3′) were used to amplify the 3′ part of the knockout construct. Both amplified constructs were digested with Eco RI (restriction sites are indicated in the primer sequences MOB1337 and MOB1338 in bold letters) and were ligated in a subsequent ligation reaction using T4 DNA ligase. The resulting ligated and purified DNA sequence was used as template for a further PCR with primer MOB1339 and MOB1313. The resulting amplification product β1-3GalT2ko

(SEQ ID NO: 18:
5′- TGGCACGATACAGTGGCATGAGATTTATCGCT-
GCCAAACTGTGGACAATGATGTTTGAAACAGTCTATTCATCACTGGTTGGCAAATTCTAT-
GTACAGGGCTAAAAGGGCCAAACTAGGCTTAACAGCAGTGATCGAGGTTCTTGAGCAGGAT-
CAGCGCAAGGGTAAGGTTGCTTAGGACCGCTTCAACCTGGTGAGTTAGACACTCAAAATAAT-
TACGAAACAGTGACATTTATAAGCTTTGTGTCGTCACTACTTTGAGCCTTCAGAGTA-
CATTTATAGGTGGTGACTTCGTTAATGATGTTAAAAATATGAGGTGAGGACATGTCTTCTTGT-
GATTAGAGTGATCACTTTGATCCTTTTGCAAACGCTGAAAGGAGTAAGTCTGATTGT-
CAACAGAAATGTTTTTGGTTGCAGCCTGGCTAATATTATTGGTCTCAGTTCAATTTTCGATG-
GAGTGGCGTACAAGTGATCCAGAAAGCAAGAATCATG-
GATTTCCTACAATTTCATTTAGATTTTCGATGTTGGTTGAGTTATGCTGATTGATTTGGGAAA-
GAGGGAGCTTAGCGTTGTATACAGGGTTCAAACACCGTAATATGAAGAGGGGTGTGAGACCAC-
CGGGTGTGCGATGTACAGGGCGGCAAAGAAACAATCTAATCAT
AGTGGCAATCATATGTTTGGTTTTTATAGCGATATTCATCCCACCGTTTCTTGAAT-
GAATTCCATAACGAAGACACCGTCTAAAGCTTCACAGGTTAGTGCAGAAATGATTGGTTCGC-
CCTCGCTATGCCAGTCAGGCTTACTGAGTTCTACTTGGATCGTTCTACTTGGATCTTTTATG-
GCTTCCTAGCAGTCGGAGGTTTCTTTCTGGTTTGAAGAAAGCCATGTATGGAACGTTTACAG-
GTTTTGGAGAAGAAAGTATCAAGCTATTTGAAAAAAGTCACTCTGGAAACTTACAGTAAAGAG-
GAACGCCGTAGTCCAGGGAACACAACAGGTGACATTGTTTCGCTGGAAGATGTGATAG-
ATCGCGCCTGGTCTGCCGGCGCCAAAGCTTGGGAAGAGCTGGAAATTGCATTCAGACAGG-
GAGAACATTTTTCGAAGAAGGACAATAATGCCAATGCAACTGCAGATCCATGCCCAGCAT-
CACTCTTTACAACAGGAAAGGAATTGGACAATTTAGGAAGGGTCTTCCCACTGCCTTGTG-
GTCTAATGTTTGGATCAGCCATAACTCTCATTGGAAAGCCACGGGAAGCTCACATG-
GAGTACAAACCGCCAATCGCCAGAGTTGGGGAAGGTGTCTCTCCATACGTCATGGTGTCCC
AGTTCATAATGGAGTTACAGGGCTTGAAGGTGGTAAAAGGTGAAGATCCTCCTA-
GAATCCTCCACATAAACCCTCGACTCCGTGGTGACTGGAGCTGGAAACCCATCAT-
TGAGCATAATACATGCTATCGAAACCAGTGGGGCCCAGCTCATCGGTGTGAAGGTTG-
GCAAGTACCTGAATACGAAGAAACCGGTGAGTGCTGGTTCCAT-
CACACTTTATCTTTTCATAGTGACACGGTTCTTTTTAGGTGTACTAGTGTTGAAAGCTGTGC-
ATGTTAAATGGTAACCCTAATCAATCTTCTCGCTAATTTTCGCATTGCAAGGTCTCCGCTGCT-
TG -3′;

Eco RI restriction site is indicated in bold letters) comprised a deletion of 148 bp in regard to the genomic sequence of the β1,3-galactosyltransferase 2 gene of Physcomitrella patens which in addition initiate a stop codon in the early 5′ part of the corresponding cDNA. Thus, resulting in a dysfunctional β1,3-galactosyltransferase gene when integrated via homologous recombination into the genome of Physcomitrella patens. This knockout construct was used for transformation of Physcomitrella patens alone or in combination with the knockout construct β1-3GalT1ko (see 2.1).

Screening of putative transformed plants was performed by PCR using appropriate primer combinations.

### 3. MALDI-TOF Mass Spectrometry

The N-glycans of glyco-engineered Physcomitrella patens strain lacking plant-specific core α1,3 fucose and β1,2 xylose residues—herein used as control—exhibit the typical structural features of plant N-glycans processed in these strains as described in Koprivova et al. 2004 Plant Biotechnol. J. 2, 517-523); i.e. no fucose in α1,3-linkage to the Asn-bound GlcNAc, and no xylose in β1,2-linkage to the βmannosyl residue, Lewis a epitopes (α1,4-fucosyl and β1,3-galactosyl residues linked to GlcNAc) as non reducing terminal elements (tab. 1). In contrast no Lewis a epitopes (α1,4-fucosyl and β1,3-galactosyl residues linked to GlcNAc) were detected on N-glycans isolated from a glyco-engineered Physcomitrella patens strain which additionally comprised targeted gene disruptions of both β1,3-galactosyltransferase 1 and β1,3-galactosyltransferase 2 genes.

TABLE 1
N-glycan structures of double knockout and tetra knockout
Physcomitrella patens strains. N-glycans were isolated from
plant material grown under same conditions (100 ml flasks, Knop
medium) residues, GF = Lewis a structure comprising fucose and
galactose (β1,3-linked), Gn = N-acetylglucosamine,
M/Man = mannose

| | Physcomitrella patens | Physcomitrella patens |
|---|---|---|
| | double knockout | tetra knockout |
| | N-glycan structures | N-glycan structures lacking core |
| | lacking core α1,3-fucose | α1,3-fucose, β1,2-xylose and β1,3- |
| | and β1,2-xylose | galactose residues (consequently |
| | residues | lacking Lewis a epitopes in total) |
| 933 | Man3 (MM) | Man3 (MM) |
| 1096 | Man4 | Man4 |
| 1137 | MGn/GnM | MGn/GnM |
| 1258 | Man5 | Man5 |
| 1299 | Man4Gn | Man4Gn |
| 1340 | GnGn | GnGn |
| 1420 | Man6 | Man6 |
| 1582 | Man7 | Man 7 |
| 1648 | (GF) Gn/Gn (GF) | |
| 1744 | Man8 | Man8 |
| 1907 | Man9 | Man9 |
| 1956 | (GF) (GF) | |

SEQ ID NO: 1
cDNA β1-3GalT1
5′AGTTGTCGATTTCTTGTTTTTGATATGTAAGGCGGTTGCCTTCGCGCCGTGCTTGATTGTAAT-
TGTAATTCAATCTGGAGTGTGAGATATATATATATATATATATATAGCGAGAGGGAGAGAGAAAGAGAGAGAGAGG-
GAGAGAGAAAGAGACAGAGAGGGAGAGAGAGAGATGGCTTGTGTATGAGGGCCATGCGAGGAGGAGGCTGT-
GTTTGTTGCCCGAAGAGATGGGATGGTTTATGTGTAGTGCAGGGGTTGGATGTGAAGCACCTGTTTGAAGGAGTCT-
GCGAGAGTTTGAAATTCCGATTCAGAGTGCGCCGATCGATGGTGCAACGTTGTTAGCAGTGATTCTTTTCGC-
CAACAGAACTGACATCATTTGGATTTTTTTTACGCGTGGATGTGCGCTCTTTTTAAAAAATTTCCGCGTGGAANA-
GAGACGGGGGTTTGTAATGGAGGCAGGCTGTGGTCATCACCCCTAGTATAGCCTGTCAAGAGAGTTCAAATTCG-
GTAATATGAAGAGGGGGTCGAGACTACCGGATATGGCGTGTACAGGGCGGCAAAGAAATGATCTTATCCTAGT-
TGCAATTGTTTGCTTGTTTTTTATGGTGATATTGATCCCACCATATCTCCAAATGAACTCACTTCCGGACAT-
TGATTCTC
CTGATTCGGACAAGAAATCATCAAGCTACTCGAAAAAAACCACTCTAGAAGCCAATAGTAAGGAGGAACGC-
CGTAGTCCGGGGAATACCACAGGGGACATTGTTTCTCTGGATGATGTGATAGATCGTGCCTGGTCTGCTGGTGC-
CAAAGCGTGGGAAGAACTGGAAACTGCGTTAAGAAATGGAGAAGGTGTCTCAAAGAATGTCAGTAATGCCACT-
GCAAATGCTGATCCGTGTCCAGCATCACTCTCTGCAGCAGGGAAAAAGTTAGACGAATTGGGTAAAGTCTTCCCCT-
TGCCCTGTGGTCTAATGTTTGGGTCAGCCATTACTCTGATTGGAAAGCCTCGAGAGGCTCACATGGAGTACAAAC-
CGCCAATCGCCAGAGTTGGGGAAGGCGTCTCTCCATATGTCATGGTTTCCCAGTTCTTAGTAGAGTTACAAGGCT-
TAAAGGTGGTGAAAGGTGAAGATCCTCCTCGAATTCTACACTTGAATCCTCGACTTCGTGGTGATTGGAGCTG-
GAAACCCATCATTGAGCACAACACTTGTTATCGGAACCAGTGGGGTCCTGCCCACCGATGCGAGGGTTGGCAAGT-
GCCTGAATACGAAGAAACTGTTGACGGTCTTCCCAAGTGCGAGAAGTGGCTTCGAGATGATGGCAAGAAACCT-
GCTTCAACGCAAAAATCTTGGTGGCTTGGAAGATTAGTTGGTCGTTCTGACAAGGAGACGCTTGAATGGGAGTAC-
CCATTATGTGAGGGTCGGGAGTTCGTTCTCACCATTCGAGCAGGTGTTGAAGGGTTTCATGTGACTATCGATG-
GTCGTCACATCAGCTCGTTTCCTTATCGTGTGGGTTACGCTGTGGAAGAAACAACGGGGATATTAGTAGCAG-
GAGACGTTGATGTGATGTCTATCACAGTGACATCCCTACCCTTAACACATCCTAGCTACTACCCTGAGT-
TAGTTTTGGAATCGGGGGACATTTGGAAGGCACCACCTGTCCCAGCTACCAAGATAGATTTATTTATTGGGATCAT-
GTCCAGCAGTAACCATTTTGCAGAACGGATGGCAGTAAGGAAGACGTG-
GTTTCAATCTAAAGCTATTCAATCTTCGCAGGCCGTGGCTCGCTTCTTTGTAGCTCTGCATGCAAACAAGGATA-
TCAATATGCAGTTGAAGAAGGAGGCAGACTATTATGGCGATATTATAATCCTGCCTTTCATCGACAGATATGATA-
TAGTGGTTCTCAAGACCGTTGAAATTTGCAAGTTTGGGGTCCAGAATGTCACAGCTAAGTATATTATGAAGTGT-
GACGATGACACTTTTGTGAGGATTGATAGCGTTCTCGAAGAGATTCGAACTACTTCAATATCACAAGGCCTTTA-
CATGGGTAGCATGAATGAGTTTCACAGGCCTCTTCGTTCTGGAAAGTGGGCCGTGACTGCCGAGGAATGGCCT-
GAGCGAATTTACCCAATATATGCTAATGGACCAGGATATATCCTGTCAGAGGATATTGTGCATTTCATTGTGGAG-
ATGAATGAGAGAGGCAGTTTGCAGTTATTTAAGATGGAGGACGTCAGTGTTGGAATATGGGTACGCGAATA-
TGCGAAGCAAGTGAAGCACGTTCAATACGAACATAGCATACGGTTTGCTCAAGCCGGTTGTATACCGAAATACT-
TGACAGCTCATTACCAATCGCCGCGTCAAATGCTGTGTCTGTGGGACAAGGTACTTGCTCATGACGATGGGAAAT-
GCTGCAACTTGTGAGGAAAATACATACAATGAATGTGTTCAACGGTCTTTACCAGACAGAATTACTTTGGGTCGG-
GAACCAGATATAGCAGACAGCTCACATTCAATTCAGCCGTGTTGATCCAGAGGGGTAATTGATAGTTTCCT-
TGTCCCCTACCCTCTCTAGAGGTGGAGATCTTACAACTTAATCAAATGATCCTCTGCAATGTCACTTGT-
CACAATACTTAGTATAGCTCAAAATTGGCCACGGATATTCAGGAATGTTCATCTTGTAAGGTCGCAGCTTGT-
GAGTAAATGGTTGGGTGGTGTCGATGGCATGGTTGCTTATCAATCCCTCTTAGCATGAGTGATCGTCAGAATCAGT-
GTTTTCGACACTCCCCGGTGGAGTATTTTTTCGATTCTCTTGATTCCACTCAAGTGGTACTAGCTTATATTTAGT-
GAGGCCTGGAACCCAAGTAGTTAGTTCAGTACGTCTGCCTTTTGCCGAAATGAGTAGAGTAATTTGTGGCAGTAGT-
TGGTGAAGAGACATGGTTAGGATTTAGTGTTCAAAATCTG 3′
SEQ ID NO: 2
cDNA Ppβ1-3GalT2
ATGAAGAGGGGTGTGAGACCACCGGGTGTGGGATGTACAGGGCGGCAAAGAAACAATCTAAT-
CATAGTGGCAATCATATGTTTGGTTTTTATAGCGATATTCATCCCACCGTTTCTTGAAAT-
GAATTCACTTCCCGATATTGATTCCCCTGTTTTGGAGAAGAAAGTAT-
CAAGCTATTTGAAAAAAGTCACTCTGGAAACTTACAGTAAAGAGGAACGCCGTAGTCCAGG-
GAACACAACAGGTGACATTGTTTCGCTGGAAGATGTGATAGATCGCGCCTGGTCTGCCGGCGC-
CAAAGCTTGGGAAGAGCTGGAAATTGCATTCAGACAGGGAGAACATTTTTCGAAGAAG-
GACAATAATGCCAATGCAACTGCAGATCCATGCCCAGCATCACTCTTTACAACAGGAAAG-
GAATTGGACAATTTAGGAAGGGTCTTCCCACTGCCTTGTGGTCTAATGTTTGGATCAGC-
CATAACTCTCATTGGAAAGCCACGGGAAGCTCACATGGAGTACAAACCGCCAATCGCCAGAGT-
TGGGGAAGGTGTCTCTCCATACGTCATGGTGTCCCAGTTCATAATGGAGTTACAGGGCT-
TGAAGGTG
GTAAAAGGTGAAGATCCTCCTAGAATCCTCCACATAAACCCTCGACTCCGTGGTGACTG-
GAGCTGGAAACCCATCATTGAGCATAATACATGCTATCGAAACCAGTGGGGCCCAGCTCATCG-
GTGTGAAGGTTGGCAAGTACCTGAATACGAAGAAACCGTGGACGGTCTTCCCAAGTGC-
GAGAAGTGGCTTCGAGGCGATGACAAAAAACCTGCTTCGACCCAAAAATCCTGGTGGCTTGG-
GCCATTAGTTGGTCATTCCGACAAGGAGACGCTTGAATGGGAGTATCCATTGTCCGAAG-
GTCGGGAGTTTGTTCTCACCATTCGAGCAGGTGTAGAAGGATTTCACTTAACTATTGATG-
GTCGGCACATCAGTTCGTTCCCTTATCGTGCGGGTTATGCTATGGAAGAAGCAACAGGAATA-
TCAGTGGCAGGAGACGTCGATGTTCTTTCGATGACAGTAACATCATTACCTTTAACA-
CATCCCAGCTACTACCCTGAGTTGGTTTTGGATTCGGGTGATATCTGGAAGGCAC-
CACCTTTACCAACAGGCAAGATAGAGTTATTTGTTGGAATCATGTCAAGCAGCAAT-
CACTTTGCAGAACGTATGGCAGTAAGAAAGACGTGGTTTCAGTCTCTGGT-
TATCCAATCCTCCCAAGCGGTGGCTCGCTTCTTTGTAGCTCTGCATGCAAACAAGGATA-
TCAATCTGCAGCTGAAGAAAGAGGCTGACTATTACGGCGATA-
TGATAATTTTACCTTTCATCGACAGATATGATATAGTGGTTCTTAAGACCGT-
TGAAATTTTCAAGTTTGGGGTCCAGAATGTTACAGTTAGCCACGTCATGAAATGTGACGAT-
GACACATTTGTAAGGATTGACAGCGTTCTTGAAGAGATTCGAACGACGTCAGTAGGACAGG-
GCCTTTACATGGGCAGCATGAATGAGTTTCATAGACCCCTTCGTTCTGGGAAGTGGGCCGT-
GACAGTTGAGGAGTGGCCTGAGCGCATTTACCCAACATACGCAAATGGTCCAGGATA-
CATCCTTTCGGAAGATATTGTGCATTTTATAGTGGAGGAGAGCAAAAGAAATAATTTGAGGT-
TATTTAAGATGGAGGACGTCAGCGTAGGTATATGGGTACGCGAGTATGCAAAGAT-
GAAGTACGTGCAATACGAGCATAGCGTACGGTTTGCTCAAGCCGGTTGTATACCTAACTACCT-
GACAGCGCACTATCAATCGCCGCGTCAAATGCTGTGTCTGTGGGACAAGGTGCTTGCTAC-
CAATGACGGCAAGTGCTGCACCTTGTGA
SEQ ID NO: 3
Genomic DNA β1-3GalT1
5′: AGTTGTCGATTTGTTGTTTTTGATATGTAAGGCGGTTGCCTTCGCGCCGTGCTTGATTGTAAT-
TGTAATTCAATCTGGAGTGTGAGATATATATATATATATATATATAGCGAGAGGGAGAGAGAAAGAGAGAGAGAGG-
GAGAGAGAAAGAGAGAGAGAGGGAGAGAGAGAGATGGCTTGTGTATGAGGGCCATGCGAGGAGGAGGCTGT-
GTTTGTTGCCCGAAGAGATGGGATGGTTTATGTGTAGTGCAGGGGTTGGATGTGAAGCACCTGTTTGAAGGAGTCT-
GCGAGAGTTTGAAATTCGGATTCAGAGTGCGGCGATCGATGGTGCAACGTTGTTAGCAGTGATTGTTTTCGC-
CAACAGAACTGACATgtaatgaatagtttcgaggcatgatcgcggtttttctcaatttgaaggggttgtttgtgg-
gtgatctatgtgcagaagtgtcactgatggtcagattcgatgcttgacaatttgatcctttgtgagtgtgcagC-
ATTTGGATTTTTTTTACGCGTGGATGTGCCCTCTTTTTAAAAAATTTCCGCGTGGAAAAGAGACGGGG-
GTTTGTAATGGAGGCAGGCTGTGGTCATCACCCCTAGTATAGCCTGTCAAGAGgtgagattgacaccctctttgct-
caattgtagatttttttccttctcagggct-
gaatcccagtttttttttttttttttttttttttttccttcttcttcaacttcgagttcgtgtctgtat-
gaagaagtccacgggttcaatgtgttaagacttaggcatttccttcagctttgcctagtggagata-
tgcgtattttttgattgtgaggattccggttcttagaccatgattggtttattacagtggt-
cattcaaatcctatttgatttgagaatgtatttacttcgttgtgttgggagatgattgttccctcgaattctat-
gcggtagctaccgcttctttcgtaatgaagacctttgaagttcacatagacttcaagaagaatgctatttgt-
gtttttgtgattgtgtgttcaagtttggtgcagtattgttaaaatttgggtgatgactaagtacactttatgcggc-
ccaagtagtcaagttgagcatttgtaaatgctgaaatgagttaggctgacggtaaatgtctgtggatgtagccta-
gtgatgtatttgatctcggcataatcttcagtgatcaatacaaataattcaagaaagaggggtcaatgtgttcct-
gcgagtaccttcgcatgttcaacgtgaactgaattatgttaattaagctgagcaacatagaccttcttgctgt-
tgacagAGTTCAAATTCGGTAATATGAAGAGGGGGTCGAGACTACCGGATATGGCGTGTACAGGGCG-
GCAAAGAAATGATCTTATCCTAGTTGCAATTGTTTGCTTGTTTTTTATGGTGATATTCATCCCACCATA-
TCTCCAAATGAACTCACTTCCGGACATTGATTCTCCTgtcgagaagctagaagatgatgatgatgct-
gtcttcacttctcatagacgtcgtaaccaagagcagatttcagttgtcactgacagtggtcagagacggacagt-
tatgccatcttcgactggtgcggaggacgtaacgaatgcaccgtctaaagattcacaggttagaccaaaagtagt-
tgacctgaaatgcatgtggtaatcaagcactcttgtccttattcgagcttttatttcttgccatcag-
gtatttttaatacttccctagtgtacgctgagtgtctacattgtgtattgaatgttccttagaat-
tgtttgtttgtttatgtttttatttttatatttctgccggctattgaggaagaatacattcaaattgttcag-
GATTCGGACAAGAAATCATCAAGCTACTCGAAAAAAACCACTCTAGAAGCCAATAGTAAGGAGGAACGC-
GGTAGTCCGGGGAATACCACAGGCGACATTGTTTCTCTGGATGATGTGATAGATCGTGCCTGGTCTGCTGGTGC-
CAAAGCGTGGGAAGAACTGGAAACTGCGTTAAGAAATGGAGAAGGTGTCTCAAAGAATGTCAGTAATGCCACT-
GCAAATGCTGATCCGTGTCCAGCATCACTCTCTGCAGCAGGGAAAAAGTTAGACGAATTGGGTAAAGTCTTCCCCT-
TGCCCTGTGGTCTAATGTTTGGGTCAGCCATTACTCTGATTGGAAAGCCTCGAGAGGCTCACATGGAGTACAAAC-
CGCCAATCGCCAGAGTTGGGGAAGGCGTCTCTCCATATGTCATGGTTTCCCAGTTCTTAGTAGAGTTACAAGGCT-
TAAAGGTGGTGAAAGGTGAAGATCCTCCTCGAATTCTACACTTGAATCCTCGACTTCGTGGTGATTGGAGCTG-
GAAACCCATCATTGAGCACAACACTTGTTATCGGAACCAGTGGGGTCCTGCCCACCGATGCGAGGGTTGGCAAGT-
GCCTGAATACGAAGAAACTCgtgagtgctgattccaccgcaccagtttgtgttttttatgctgacactatgcttct-
caggtttgtagacgttaagagctgtgtaggttccgtggtacttcgaattggcacttgccacttctctcat-
tgtaagttggtaaatgtctgcatgagcaataaattccaacactggatgtgtattttctgaaatgattcgttttct-
tgtagTTGACGGTCTTCCCAAGTGCGAGAAGTGGCTTCGAGATGATGGCAAGAAACCTGCTTCAACGCAAAAATCT-
TGGTGGCTTGGAAGATTAGTTGGTCGTTCTGACAAGGAGACGCTTGAATGGGAGTACCCATTATCTGAGGGTCGG-
GAGTTCGTTCTCACCATTCGAGCAGGTGTTGAAGGGTTTCATGTGACTATCGATGGTCGTCACAT-
CAGCTCGTTTCCTTATCGTGTGgtaagttgaaaatgctatgttaacatataatgctaaagttgacctcat-
gtctttcttttttctttttttcttttttattttctggagggggggggggtaatgcaaat-
caactctaaaattttagtataccagttaaattattcatttcaaatataacaatacaaataca-
catctttttaatttgtattttttgatccctctcctcctctactaaaattaataatatagcaacattttggtac-
tacgaaagttcatttgtattgcttcatgtcgaagatttattcaaaatttctatccctcgtgtttctgaattacat-
tatcaacaatggaataacaataatgacggccccatccttcagacaccaggaacattacctataccagactacgtct-
gggtaagtctgaagaattaattataaccaagaaactagttgtattcactgtttttctttttacgcccat-
gcgatttatcgaagtcttcttcaatttcttattattcttctttattattttaagtttttaat-
tatttttaaagcaacgaattgataaataaataacatattaat-
gtttttaactttaaagtttttttcccgtatttagtataagatttcgtcaaaacgattaggtgattagatcgaacat-
tatctaattgcactctacttatatgatatgaagagtaatttctcttagcagaagctacatcctgctatttccttgg-
gaaacccgattaggtctttcaaatcacccctgcttcctctataagtgtaccatgattgaggttcgttagggc-
attagtttaagggtatcgttgtgatgtgtgtctagttagtcttaaaatctgtgcaaatcgattcat-
taacaactcttttctgtagtgttttgttttgagaactgctatttatcttccattgtgcagGGTTACGCTGTG-
GAAGAAACAACGGGGATATTAGTAGCAGGAGACGTTGATGTGATGTCTATCACAGTGACATCCCTACCCTTAACA-
CATCCTAGCTACTACCCTGAGTTAGTTTTGGAATCGGGGGACATTTGGAAGGCACCACCTGTGCCAGCTAC-
CAAGATAGATTTATTTATTGGGATCATGTCCAGCAGTAACCATTTTGCAGAAGGGATGGCAGTAAGGAAGACGTG-
GTTTCAATCTAAAGCTATTCAATCTTCGCAGGCCGTGGCTCGCTTCTTTGTAGCTCTGgtacttcctcctat-
caaatctcattaactttcgaattattagtgatcatctacataagtggtctgttgattgctgaaaggtggctgt-
tgcgtgcctttgcgtaatgactttccaaattcatttagaacagtggaaacataatttgtgtgttgcgt-
tgcgtatttaactttttcggtgaatgtcttattgaattgtgatgtagCATGCAAACAAGGATATCAATATGCAGT-
TGAAGAAGGAGGCAGACTATTATGGCGATATTATAATCCTGCCTTTCATCGACAGATATGATATAGTGGTTCT-
CAAGACCGTTGAAATTTGCAAGTTTGGGgtacgtgtgtcgaataatggcttcaaagctttgtgacggtgtct-
gcaatttggggatggtgataatgaggcttgataccaactgaaggttaggtgacttttaacactaggttctgct-
tactgtgcagGTCCAGAATGTCACAGCTAAGTATATTATGAAGTGTGACGATGACACTTTTGTGAGGAT-
TGATAGCGTTCTCGAAGAGATTCGAACTACTTCAATATCACAAGGCCTTTACATGGGTAGCATGAAT-
GAGTTTCACAGGCCTCTTCGTTCTGGAAAGTGGGCCGTGACTGCCGAGgtatttttatttttatttttg-
gcttttgtcgggaacgtgagagaaaccaagatgaatataatcacgatgttgttttttattgcaaggatttatttg-
atgctcttgagaaatctgtggtagccataccactcaatttggatactagatgtgttcgtccttatgtataaaaat-
gaaacatgtgcttttcaggaagattaattcagtttgacttgtacgtctagttagattgatggtgatgaaacaagag-
gattatctcgcgaattgacaagtgggttgcttggacagGAATGGCCTGAGCGAATTTACCCAATATATGCTAATG-
GACCAGGATATATCCTGTCAGAGGATATTGTGCATTTCATTGTGGAGATGAATGAGAGAGGCAGTTTGCAGgtag-
gttcttttagaactgtgtcgtcgctattacacgtctacaagttttaaaaattagaaactttcttgttg-
gcaaatttccatccaggaatctttttgcaccgcaagttcgtaataggagtcggtacattctgtgtgtgt-
gcatcgtttgttaaatgcatttttcaattttcttttgcttaaaatatctctgttgtcgatatctcctcatgatct-
tgcattgtgaacatgagaagatatgaaatgtgaactcaatattcttctatgatcatgtgcagTTATTTAAGATG-
GAGGACGTCAGTGTTGGAATATGGGTACGCGAATATGCGAAGCAAGTGAAGCACGTTCAATACGAA-
CATAGCATACGGTTTGCTCAAGCCGGTTGTATACCGAAATACTTGACAGCTCATTACCAATCGCCGCGTCAAAT-
GCTGTGTCTGTGGGACAAGGTACTTGCTCATGACGATGGGAAATGCTGCAACTTGTGAGGAAAATACATACAAT-
GAATGTGTTCAACGGTCTTTACCAGACAGAATTACTTTGGGTCGGGAACCAGATATAGCAGACAGCTCA-
CATTCAATTCAGCCGTGTTGATCCAGAGGGGTAATTGATAGTTTCCTTGTCCCCTACCCTCTCTAGAGGTGGAG-
ATCTTACAACTTAATCAAATGATCCTCTGCAATGTCACTTGTCACAATACTTAGTATAGCTCAAAATTGGCCACG-
GATATTCAGGAATGTTCATCTTGTAAGGTCGCAGCTTGTGAGTAAATGGTTGGGTGGTGTCGATGGCATGGTTGCT-
TATCAATCCCTCTTAGCATCAGTGATCGTCAGAATCAGTGTTTTCGACACTCCCCGGTG-
GAGTATTTTTTCGATTCTCTTGATTCCACTCAAGTGGTACTAGCTTATATTTAGTGAGGCCTGGAACCCAAGTAGT-
TAGTTCAGTACGTCTGCCTTTTGCCGAAATGAGTAGAGTAATTTGTGGCAGTAGTTGGTGAAGAGACATGGTTAG-
GATTTAGTGTTCAAAATCTG 3′
SEQ ID NO: 4
Genomic DNA Ppβ1-3GalT2
ATGAAGAGGGGTGTGAGACCACCGGGTGTGGGATGTACAGGGCGGCAAAGAAACAATCTAATCATAGTGGCAAT-
CATATGTTTGGTTTTTATAGCGATATTCATCCCACCGTTTCTTGAAATGAATTCACTTCCCGATATTGATTCCCCT-
gtgtataggttagaaggtattaacttcgcttcacatagacgtcgctatcaagaacaggattcacgtgtcagt-
tacagtggctatggacagccagatatgccatcaactggtgatgaagacataacgaagacac-
cgtctaaagcttcacaggttagtgcagaaatgattggttcgccctcgctatgccagtcaggcttactgagttc-
tacttggatcgttctacttggatcttttatggcttcctagcagtcggaggtttctttctggtttgaagaaagccat-
gtatggaacgtttacagGTTTTGGAGAAGAAAGTATCAAGCTATTTGAAAAAAGTCACTCTGGAAACT-
TACAGTAAAGAGGAACGCCGTAGTCCAGGGAACACAACAGGTGACATTGTTTCGCTGGAAGATGTGATAG-
ATCGCGCCTGGTCTGCCGGCGCCAAAGCTTGGGAAGAGCTGGAAATTGCATTCAGACAGGGAGAA-
CATTTTTCGAAGAAGGACAATAATGCCAATGCAACTGCAGATCCATGCCCAGCATCACTCTTTACAACAGGAAAG-
GAATTGGACAATTTAGGAAGGGTCTTCCCACTGCCTTGTGGTCTAATGTTTGGATCAGCCATAACTCTCATTG-
GAAAGCCACGGGAAGCTCACATGGAGTACAAACCGCCAATCGCCAGAGTTGGGGAAGGTGTCTCTCCATACGTCAT-
GGTGTCCCAGTTCATAATGGAGTTACAGGGCTTGAAGGTGGTAAAAGGTGAAGATCCTCCTAGAATCCTCCA-
CATAAACCCTCGACTCCGTGGTGACTGGAGCTGGAAACCCATCATTGAGCATAATACATGCTATCGAAACCAGT-
GGGGCCCAGCTCATGGGTGTGAAGGTTGGCAAGTACCTGAATACGAAGAAACCGgtgagtgctggttccat-
cacactttatcttttcatagtgacacggttctttttaggtgtactagtgttgaaagctgtgcatgttaaatg-
gtaaccctaatcaatcttctcgctaattttcgcattgcaaggtctccgctgcttggacaatcagcactctaacat-
tggctgtatttactgaaatgattctttactttgtagTGGACGGTCTTCCCAAGTGCGAGAAGTGGCTTCGAGGCG-
ATGACAAAAAACCTGCTTCGACCCAAAAATCCTGGTGGCTTGGGCGATTAGTTGGTCATTCCGACAAGGAGACGCT-
TGAATGGGAGTATCCATTGTCCGAAGGTCGGGAGTTTGTTCTCACCATTCGAGCAGGTGTAGAAGGATTTCACT-
TAACTATTGATGGTCGGCACATCAGTTCGTTCCCTTATCGTGCGgtgagttgaaaatactagtttgatatctaatg-
atgaggtttaccgcaggtatatttggtctcattgtcaagtgtgtgtgtgtgtgt-
tgtttttcttttttccttttcattttctgaatcataatgataagaaatcaattctatgaaacttagcgtcaata-
ttttaaagttttattgtttttgtttgtttttatttttttgtgttttgtgttttgtgtttatttcacaatacaatgt-
taacaatggaatagaaacaatgatggtcccacctcacagacaccaggtacactacctacaccagactgcgtct-
gagtaagtttaagaaacagcaaccaccaacaatctgattgtaaattctaaattccttctccaccagaaaaccatgt-
gatccgtcttgcagttctgcttgcactctacctatatgatccaaagagtaattcctcttaacaggagttataacct-
gctggggttttgaaaataccgatgagttcaaattgtaaacaaaccccggatctatttcaagggtatgaagggct-
tagctttgtttaagaataaggtcaagagtatctgtgtggtgagcatcccaaaatggatgcaaatttgttaattg-
gcaactgttttctgtggtatgttttgtgacgcactatttattgtgtattgtgcagGGTTATGCTATG-
GAAGAAGCAACAGGAATATCAGTGGCAGGAGACGTCGATGTTCTTTCGATGACAGTAACATCATTACCTTTAACA-
CATCCCAGCTACTACCCTGAGTTGGTTTTGGATTCGGGTGATATCTGGAAGGCACCACCTTTACCAACAG-
GCAAGATAGAGTTATTTGTTGGAATCATGTCAAGGAGCAATCACTTTGCAGAACGTATGGCAGTAAGAAAGACGTG-
GTTTCAGTCTCTGGTTATCCAATCCTCCCAAGCGGTGGCTCGCTTCTTTGTAGCTCTGgtacttgtcat-
tatactcttttttcgtgccaagtatcgtgaactcgggaatatttaaaaagtgcaaacaacaagtgagctgttaat-
tgctgaaaattggtgttataagtcttgatgcagtgaccttccagattgaccaagtatatcagacct-
tagaatttgaacagcactacttacttaccatttttaatgaatcccttgttgggttgtgatgcagCATGCAAACAAG-
GATATCAATCTGCAGCTGAAGAAAGAGGCTGACTATTACGGCGATATGATAATTTTACCTTTCATCGACAGATA-
TGATATAGTGGTTCTTAAGACCGTTGAAATTTTCAAGTTTGGGgtaagcgaat-
taaaatttgtagtatttacaaagtaatatttttaaacgttgtgaggacatctgcaacttgatatatttctttcgt-
gaggttcgatgctgattaaagcttaggtgatttaaaagcacggtgttgcttgctatgcagGTCCAGAATGT-
TACAGTTAGCCACGTCATGAAATGTGACGATGACACATTTGTAAGGATTGACAGGGTTCTTGAA-
GAGATTCGAACGACGTCAGTAGGACAGGGCCTTTACATGGGCAGCATGAATGAGTTTCATAGACCCCTTCGTTCT-
GGGAAGTGGGCCGTGACAGTTGAGgtaattttccctgtaccaaattatccaagattttcgtaaccattgtgtgcct-
tattcatttcttctgaaatctcaagaaaaatgaaaaatgcttgagaaacgctcgtagccgtatcacattat-
gcgaattccaaaaaagaatgtggaacaaaagttcttgtgaaaataattgatatgttcaaattgtacacatttat-
gcactaagataagatatgtgcaaatagtgccttccagtggtctagaaaatgcttgtttttttttg-
gaagctttaactttatttagcttgaacatcttgtttgagggttggtgaccaagtaagaag-
gtccatacaagacaataaatggattggttcgtgcatgtacagGAGTGGCCTGAGCGCATTTACCCAA-
CATACGCAAATGGTCCAGGATACATCCTTTCGGAAGATATTGTGCATTTTATAGTGGAGGA-
GAGCAAAAGAAATAATTTGAGGgtgcgtttttcatagctgtgtcctggtgattaaatgccccatgttcaacat-
tgaaaccttcatcttggacagttttccatccatgtatctcctgtcattataattgcattatagaactgttcgcgt-
gtacatttctttcctgttcctctttttcattttctttttctcttcttttcttcatttacttctcctcttgtcgat-
gctttctgttgaccttatattgtggatatgtatctcttcagtactacggagacgatatgaaacataagtttgata-
ttcttctgtgataaagcgcagTTATTTAAGATGGAGGACGTCAGGGTAGGTATATGGGTACGCGAGTATGCAAAG-
ATGAAGTACGTGCAATACGAGCATAGCGTACGGTTTGCTCAAGCCGGTTGTATACCTAACTACCT-
GACAGCGCACTATCAATCGCCGCGTCAAATGCTGTGTCTGTGGGACAAGGTGCTTGCTACCAATGACGGCAAGT-
GCTGCACCTTGTGA
SEQ ID NO. 24
cDNA β1, 3GalT1 alternative splice variant
165 nucleotide splice insert shown in bold letters (nt471-635)
ATGCGAGGAGGAGGCTGTGTTTGTTGCCCGAAGAGATGGGATGGTTTATG
TGTAGTGCAGGGGTTGGATGTGAAGCACCTGTTTGAAGGAGTCTGCGAGA
GTTTGAAATTCGGATTCAGAGTGCGGCGATCGATGGTGCAACGTTGTTAG
CAGTGATTGTTTTCGCCAACAGAACTGACATCATTTGGATTTTTTTTACG
CGTGGATGTGCCCTCTTTTTAAAAAATTTCCGCGTGGAAAAGAGACGGGG
GTTTGTAATGGAGGCAGGCTGTGGTCATCACCCCTAGTATAGCCTGTCAA
GAGAGTTCAAATTCGGTAATATGAAGAGGGGGTCGAGACTACCGGATATG
GCGTGTACAGGGCGGCAAAGAAATGATCTTATCCTAGTTGCAATTGTTTG
CTTGTTTTTTATGGTGATATTCATCCCACCATATGTCCAAATGAACTGAC
TTCCGGACATTGATTCTCCTGTCGAGAAGCTAGAAGATGATGATGATGCT
GTCTTCACTTCTCATAGACGTCGTAACCAAGAGCAGATTTCAGTTGTCAC
TGACAGTGGTCAGAGACGGACAGTTATGCCATCTTCGACTGGTGCGGAGG
ACGTAACGAATGCACCGTCTAAAGATTCACAGGATTCGGACAAGAAATCA
TCAAGCTACTCGAAAAAAACCACTCTAGAAGGCAATAGTAAGGAGGAACG
CCGTAGTCCGGGGAATACCACAGGCGACATTGTTTCTCTGGATGATGTGA
TAGATCGTGCCTGGTCTGCTGGTGCCAAGCGTGGGAAGAACTGGAAAACT
GCGTTAAGAAATGGAGAAGGTGTCTCAAAGAATGTCAGTAATGCCACTGC
AAATGCTGATCCGTGTCCAGCATCACTCTCTGCAGCAGGGAAAAAGTTAG
ACGAATTGGGTAAAGTCTTCCCCTTGCCCTGTGGTCTAATGTTTGGGTCA
GCCATTACTCTGATTGGAAAGCCTCGAGAGGCTCACATGGAGTACAAACC
GCCAATCGCCAGAGTTGGGGAAGGCGTCTCTCCATATGTCATGGTTTCCC
AGTTCTTAGTAGAGTTACAAGGCTTAAAGGTGGTGAAAGGTGAAGATCCT
CCTCGAATTCTACACTTGAATCCTCGACTTCGTGGTGATTGGAGCTGGAA
ACCCATCATTGAGCACAACACTTGTTATCGGAACCAGTGGGGTCCTGCCC
ACCGATGCGAGGGTTGGCAAGTGCCTGAATACGAAGAAACTGTTGACGGT
CTTCCCAAGTGCGAGAAGTGGCTTCGAGATGATGGCAAGAAACCTGCTTC
AACGCAAAAATCTTGGTGGCTTGGAAGATTAGTTGGTCGTTGTGACAAGG
AGACGCTTGAATGGGAGTACCCATTATCTGAGGGTCGGGAGTTCGTTCTC
ACCATTCGAGCAGGTGTTGAAGGGTTTCATGTGACTATCGATGGTCGTCA
CATCAGCTCGTTTCCTTATCGTGTGGGTTACGCTGTGGAAGAAACAACGG
GGATATTAGTAGCAGGAGACGTTGATGTGATGTCTATCACAGTGACATCC
CTACCCTTAACACATCCTAGCTACTACCCTGAGTTAGTTTTGGAATCGGG
GGACATTTGGAAGGCACCACCTGTCCCAGCTACCAAGATAGATTTATTTA
TTGGGATCATGTCCAGCAGTAACCATTTTGCAGAACGGATGGCAGTAAGG
AAGACGTGGTTTCAATCTAAAGCTATTCAATCTTCGCAGGCCGTGGCTCG
CTTCTTTGTAGCTCTGCATGCAAACAAGGATATCAATATGCAGTTGAAGA
AGGAGGCAGACTATTATGGCGATATTATAATCCTGCCTTTCATCGACAGA
TATGATATAGTGGTTCTCAAGACCGTTGAAATTTGCAAGTTTGGGGTCCA
GAATGTCACAGCTAAGTATATTATGAAGTGTGACGATGACACTTTTGTGA
GGATTGATAGCGTTCTCGAAGAGATTCGAACTACTTCAATATCACAAGGC
CTTTACATGGGTAGCATGAATGAGTTTCACAGGCCTCTTCGTTCTGGAAA
GTGGGCCGTGACTGCCGAGGAATGGCCTGAGCGAATTTACCCAATATATG
CTAATGGACCAGGATATATCCTGTCAGAGGATATTGTGCATTTCATTGTG
GAGATGAATGAGAGAGGCAGTTTGCAGTTATTTAAGATGGAGGACGTCAG
TGTTGGAATATGGGTACGCGAATATGCGAAGCAAGTGAAGCACGTTCAAT
ACGAACATAGCATACGGTTTGCTCAAGCCGGTTGTATACCGAAATACTTG
ACAGCTCATTACCAATCGCCGCGTCAAATGCTGTGTCTGTGGGACAAGGT
ACTTGCTGATGACGATGGGAAATGCTGCAACTTGTGA
SEQ ID NO: 25
cDNA β1, 3-GalT2 alternative splice variant
150 nucleotide splice insert shown in bold letters (nt151-300)
ATGAAGAGGGGTGTGAGACCACCGGGTGTGGGATGTACAGGGCGGCAAAG
AAACAATCTAATCATAGTGGCAATCATATGTTTGGTTTTTATAGCGATAT
TCATCCCACCGTTTCTTGAAATGAATTCACTTCCCGATATTGATTCCCCT
GTGTATAGGTTAGAAGGTATTAACTTCGCTTCACATAGACGTCGCTATCA
AGAACAGGATTCACGTGTCAGTTACAGTGGCTATGGACAGCCAGATATGC
CATCAACTGGTGATGAAGACATAACGAAGACACCGTCTAAAGCTTCACAG
GTTTTGGAGAAGAAAGTATCAAGCTATTTGAAAAAAGTCACTCTGGAAAC
TTACAGTAAAGAGGAACGCCGTAGTCCAGGGAACACAACAGGTGACATTG
TTTCGCTGGAAGATGTGATAGATCGCGCCTGGTCTGCCGGCGCCAAAGCT
TGGGAAGAGCTGGAAATTGCATTCAGACAGGGAGAACATTTTTCGAAGAA
GGACAATAATGCCAATGCAACTGCAGATCCATGCCCAGCATCACTCTTTA
CAACAGGAAAGGAATTGGACAATTTAGGAAGGGTCTTCCCACTGCCTTGT
GGTCTAATGTTTGGATCAGCCATAACTCTCATTGGAAAGCCACGGGAAGC
TCACATGGAGTACAAACCGCCAATCGCCAGAGTTGGGGAAGGTGTCTCTC
CATACGTCATGGTGTCCCAGTTCATAATGGAGTTACAGGGCTTGAAGGTG
GTAAAAGGTGAAGATCCTCCTAGAATCCTCCACATAAACCCTCGACTCCG
TGGTGACTGGAGCTGGAAACCCATCATTGAGCATAATACATGCTATCGAA
ACCAGTGGGGCCCAGCTCATCGGTGTGAAGGTTGGCAAGTACCTGAATAC
GAAGAAACCGTGGACGGTCTTCCCAAGTGCGAGAAGTGGCTTCGAGGCGA
TGACAAAAAACCTGCTTCGACCCAAAAATCCTGGTGGCTTGGGCGATTAG
TTGGTCATTCCGACAAGGAGACGCTTGAATGGGAGTATCCATTGTCCGAA
GGTCGGGAGTTTGTTCTCACCATTCGAGCAGGTGTAGAAGGATTTCACTT
AACTATTGATGGTCGGCACATCAGTTCGTTCCCTTATCGTGCGGGTTATG
CTATGGAAGAAGCAACAGGAATATCAGTGGCAGGAGACGTCGATGTTCTT
TCGATGACAGTAACATCATTACCTTTAACACATCCCAGCTACTACCCTGA
GTTGGTTTTGGATTCGGGTGATATCTGGAAGGCACCACCTTTACCAACAG
GCAAGATAGAGTTATTTGTTGGAATCATGTCAAGCAGCAATCACTTTGCA
GAACGTATGGCAGTAAGAAAGACGTGGTTTCAGTCTCTGGTTATCCAATC
CTCCCAAGCGGTGGCTCGCTTCTTTGTAGCTCTGCATGCAAACAAGGATA
TCAATCTGCAGCTGAAGAAAGAGGCTGACTATTACGGCGATATGATAATT
TTACCTTTCATCGACAGATATGATATAGTGGTTCTTAAGACCGTTGAAAT
TTTCAAGTTTGGGGTCCAGAATGTTACAGTTAGCCACGTCATGAAATGTG
ACGATGACACATTTGTAAGGATTGACAGCGTTCTTGAAGAGATTCGAACG
ACGTCAGTAGGACAGGGCCTTTACATGGGCAGCATGAATGAGTTTCATAG
ACCCCTTCGTTCTGGGAAGTGGGCCGTGACAGTTGAGGAGTGGCCTGAGC
GCATTTACCCAACATACGCAAATGGTCCAGGATACATCCTTTCGGAAGAT
ATTGTGGATTTTATAGTGGAGGAGAGCAAAAGAAATAATTTGAGGTTATT
TAAGATGGAGGACGTCAGCGTAGGTATATGGGTACGCGAGTATGCAAAGA
TGAAGTACGTGCAATACGAGCATAGCGTACGGTTTGCTCAAGCCGGTTGT
ATACCTAACTACCTGACAGCGCACTATCAATCGCCGCGTCAAATGCTGTG
TCTGTGGGACAAGGTGCTTGCTACCAATGACGGCAAGTGCTGCACCTTGT
GA

1.-32. (canceled)

33. A DNA molecule comprising a sequence coding for a plant protein having β1,3-galactosyltransferase activity (β1,3-GalT activity) or being complementary to such a sequence, wherein the sequence is further defined as:
a sequence:
of SEQ ID NO: 1 comprising an open reading frame from base pair 513 to base pair 2417, having at least 50% identity with this sequence, or degenerated to this sequence due to the genetic code;
of SEQ ID NO: 2 comprising an open reading frame from base pair 1 to base pair 1902, having at least 50% identity with this sequence, or degenerated to this sequence due to the genetic code;
of SEQ ID NO: 24 comprising an open reading frame from base pair 321 to base pair 2387, having at least 50% identity with this sequence, or degenerated to this sequence due to the genetic code; or
of SEQ ID NO: 25 comprising an open reading frame from base pair 1 to 2052, having at least 50% identity with this sequence, or degenerated to this sequence due to the genetic code;
a sequence having at least 20% overall identity to a sequence of SEQ ID NO: 1, SEQ ID NO: 2, SEQ ID NO: 24 or SEQ ID NO: 25 and having at least 80% identity to a sequence of seven conserved domains of SEQ ID NO: 1 or SEQ ID NO: 2 encoding amino acids 387-392 (DLFIGI—SEQ ID NO: 28 or ELFVGI—SEQ ID NO: 29), 402-409 (RMAVRKTW—SEQ ID NO: 30), 425-428 (FVAL—SEQ ID NO: 31), 455-465 (DRYDIVVLKTV—SEQ ID NO: 32), 479-489 (YIMKCDDDTFV—SEQ ID NO: 33 or HVMKCDDDTFV—SEQ ID NO: 34), 536-548 (YPIYANGPGYILS—SEQ ID NO: 35 or YPTYANGPGYILS—SEQ ID NO: 36) and 570-576 (EDVSVGI—SEQ ID NO: 37) of the protein of SEQ ID NO: 19 or SEQ ID NO: 20, or comprising a sequence which is degenerated to one of these sequences due to the genetic code; or
a sequence having at least 20% overall identity to a sequence of SEQ ID NO: 1, SEQ ID NO: 2, SEQ ID NO: 24 or SEQ ID NO: 25 and encoding at least 95% of the conserved amino acids of the seven conserved domains of SEQ ID NO: 1 or SEQ ID NO: 2 selected from amino acids 388 (L), 402 (R), 404 (A), 406 (R), 408 (T), 409 (W), 425 (F), 455 (D), 457 (Y), 463 (K), 464 (T), 481 (M), 482 (K), 484 (D), 486 (D), 488 (F), 489 (V), 536 (Y), 537 (P), 542 (G), 544 (G), 545 (Y), 548 (S), 570 (E), 571 (D), 572 (V), 575 (G) and 576 (I) of the protein of SEQ ID NO: 19 or SEQ ID NO: 20, or comprising a sequence which is degenerated to one of these sequences due to the genetic code; or
a partial sequence of any of the above.

34. The DNA molecule of claim 33, further defined as a partial sequence having at least 80% identity with a sequence of and having at least 80% identity with a sequence of SEQ ID NO: 1, SEQ ID NO: 2, SEQ ID NO: 24 or SEQ ID NO: 25 or complementary thereto and a size of 15 to 300 base pairs.

35. The DNA molecule of claim 34, further defined as having a size of 20 to 50 base pairs.

36. The DNA molecule of claim 33, further defined as coding for a protein having GlcNAc-β1,3-galactosyltransferase activity.

37. The DNA molecule of claim 33, further defined as coding for a protein having activity in respect to the transfer of galactose from UDP-galactose to non-reducing GlcNAc residues.

38. The DNA molecule of claim 33, further defined as coding for a protein having activity in respect to the transfer of galactose from UDP-galactose to non-reducing GlcNAc residues of N-glycan structures linked to proteins.

39. The DNA molecule of claim 33, further defined as comprising at least 70% identity with one of the sequences of to SEQ ID NO: 1, SEQ ID NO: 2, SEQ ID NO: 24 or SEQ ID NO: 25 or is degenerated due to the genetic code or is complementary thereto.

40. The DNA molecule of claim 39, further defined as comprising at least 80% identity with one of the sequences of to SEQ ID NO: 1, SEQ ID NO: 2, SEQ ID NO: 24 or SEQ ID NO: 25 or is degenerated due to the genetic code or is complementary thereto.

41. The DNA molecule of claim 40, further defined as comprising at least 90% identity with one of the sequences of to SEQ ID NO: 1, SEQ ID NO: 2, SEQ ID NO: 24 or SEQ ID NO: 25 or is degenerated due to the genetic code or is complementary thereto.

42. The DNA molecule of claim 33, further defined as having a sequence according to SEQ ID NO: 1 with an open reading frame from base pair 513 to base pair 2417 or a sequence according to SEQ ID NO: 2 with an open reading frame from base pair 1 to base pair 1902, or has at least 50% identity with at least one of the above sequences, or comprises a sequence which is degenerated to the above sequences due to the genetic code, with the sequences coding for plant proteins having β1,3-galactosyltransferase activity (β1,3-GalT activity) or being complementary thereto.

43. The DNA molecule of claim 33, further defined as covalently associated with a detectable marker substance.

44. The DNA molecule of claim 33, further defined as comprising a transmembrane domain encoding DNA sequence operably linked to a heterologous protein.

45. The DNA molecule of claim 44, wherein the heterologous protein is an enzyme.

46. The DNA molecule of claim 45, wherein in the heterologous protein is involved in posttranslational modification of proteins.

47. An expression vector comprising a DNA molecule of claim 33.

48. An expression vector comprising a DNA molecule of claim 33 inversely oriented with respect to a promoter.

49. A DNA molecule coding for a ribozyme comprising two sequence sections, each of which has a length of at least 10 to 15 base pairs and which are complementary to the sequence sections of a DNA molecule of claim 33, wherein the ribozyme can complex with and cut mRNA transcribed by a natural β1,3-GalT molecule.

50. A biologically functional vector comprising a DNA molecule of claim 49.

51. A method of expressing β1,3-galactosyltransferase comprising:
obtaining a DNA molecule of claim 33;
cloning the DNA molecule into a vector; and
transfecting the vector into a host cell;
wherein the host cell expresses active β1,3 galactosyltransferase.

52. The method of claim 51, wherein the host cell is comprised in a tissue or a host comprising host cells selection and amplification of transfected host cells.

53. The method of claim 51, wherein the DNA molecule of claim 33 lacks at least a transmembrane encoding sequence.

54. A protein expressed according to the method of claim 51, further defined as being active and able to elongate N-glycans of glycoproteins in vitro and/or in vivo.

55. A DNA vector comprising a molecule with a nucleic acid sequence according to SEQ ID NO: 3 or SEQ ID NO: 4.

56. A method of preparing a recombinant cell and/or plant containing a recombinant cell wherein production of β1,3-galactosyltransferase is suppressed or stopped, comprising:
obtaining a DNA molecule of claim 33 that comprises a deletion, insertion and/or substitution mutation; and
inserting the DNA molecule into a host cell or plant.

57. The method of claim 56, wherein the DNA molecule is inserted into the cell or plant at a genomic position of the non-mutated, homologous sequence of the cell or plant.

58. A recombinant plant or plant cell comprising a DNA molecule of claim 33 that comprises a deletion, insertion and/or substitution mutation and suppressed or stopped endogenous β1,3-galactosyltransferase production.

59. The recombinant plant or plant cell of claim 58, wherein the DNA molecule is at a genomic position of the non-mutated, homologous sequence of the cell or plant.

60. A peptide nucleic acid (PNA) molecule, comprising a sequence of a DNA of claim 33 or complementary thereto.

61. A method of producing a plant or cell having blocked expression of β1,3-galactosyltransferase at the transcription or translation level comprising:
obtaining a PNA molecule of claim 60; and
inserting the PNA molecule into a plant or cell.

62. The method of claim 61, further defined as method of producing a plant or cell producing a recombinant glycoprotein further comprising transfecting the plant or cell with a DNA molecule that codes for the glycoprotein.

63. The method of claim 62, wherein the recombinant glycoprotein is further defined as a human glycoprotein.

64. A method of producing recombinant glycoproteins comprising:
obtaining a plant or cell produced by the method of claim 62; and
growing or culturing the plant or cell under conditions leading to the production of recombinant glycoproteins.

65. A method of producing glycoproteins with N-glycans, comprising the in vitro or in vivo elongation of the N-glycan of a glycoprotein with an active β1,3-galactosyltransferase encoded by a DNA molecule of claim 33.

66. A method of selecting DNA molecules coding for a β1,3-galactosyltransferase comprising:
obtaining a sample;
obtaining a DNA molecule of claim 43;
adding the DNA molecule to the sample; and
binding the DNA molecule to DNA coding for a β1,3-galactosyltransferase.

67. The method of claim 66, wherein the sample comprises genomic DNA of a plant organism.
